SEQUENCE LISTING (73 sequences)

SEQ ID NO: 1 - protein, 117 aa, Artificial Sequence
  VH Amino acid Sequence of 16E3H3, 16E3H3 M1 (humanized)
QVQLVQSGAEVKKPGASVKVSCKASGYPFTTYGMNWVKQAPGQGLEWMGWIDTYSGVPTYADDFKGRVTMTRDTSISTAYMELSRLRSDDTAVYFCARRSFWYFDVWGAGTTVTVSS

SEQ ID NO: 2 - protein, 5 aa, Artificial Sequence
  CDR H1 Sequence of 16E3H3, 16E3H3 M1
TYGMN

SEQ ID NO: 3 - protein, 17 aa, Artificial Sequence
  CDR H2 Sequence of 16E3H3, 16E3H3 M1
WIDTYSGVPTYADDFKG

SEQ ID NO: 4 - protein, 8 aa, Artificial Sequence
  CDR H3 Sequence of 16E3H3, 16E3H3 M1
RSFWYFDV

SEQ ID NO: 5 - protein, 113 aa, Artificial Sequence
  VL Amino acid Sequence of 16E3H3 (humanized)
DVVMTQSPLSLPVTLGQPASISCRSSQSLVHSNGNTYLHWYLQRPGQSPRLLIYKVSNRFSGVPDRFSGSGSGTDFTLKISRVEAEDVGVYFCSQSTHVPWTFGGGTKVEIKR

SEQ ID NO: 6 - protein, 113 aa, Artificial Sequence
  VL Amino acid Sequence of 16E3H3 M1 (humanized)
DVVMTQSPLSLPVTLGQPASISCRSSQSLVHSNANTYLHWYLQRPGQSPRLLIYKVSNRFSGVPDRFSGSGSGTDFTLKISRVEAEDVGVYFCSQSTHVPWTFGGGTKVEIKR

SEQ ID NO: 7 - protein, 16 aa, Artificial Sequence
  CDR L1 Sequence of 16E3H3
RSSQSLVHSNGNTYLH

SEQ ID NO: 8 - protein, 16 aa, Artificial Sequence
  CDR L1 Sequence of 16E3H3 M1
RSSQSLVHSNANTYLH

SEQ ID NO: 9 - protein, 7 aa, Artificial Sequence
  CDR L2 Sequence of 16E3H3, 16E3H3 M1
KVSNRFS

SEQ ID NO: 10 - protein, 9 aa, Artificial Sequence
  CDR L3 Sequence of 16E3H3, 16E3H3 M1
SQSTHVPWT

SEQ ID NO: 11 - protein, 447 aa, Artificial Sequence
  Full sequence of Heavy Chain of B7-H4 IgG1 (16E3H3 and 16E3H3 M1)
QVQLVQSGAEVKKPGASVKVSCKASGYPFTTYGMNWVKQAPGQGLEWMGWIDTYSGVPTYADDFKGRVTMTRDTSISTAYMELSRLRSDDTAVYFCARRSFWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 30 aa, Artificial Sequence
  Framework 1 sequence of B7-H4 IgG1 (16E3H3 and 16E3H3 M1)
QVQLVQSGAEVKKPGASVKVSCKASGYPFT

SEQ ID NO: 13 - protein, 14 aa, Artificial Sequence
  Framework 2 sequence of B7-H4 IgG1 (16E3H3 and 16E3H3 M1)
WVKQAPGQGLEWMG

SEQ ID NO: 14 - protein, 32 aa, Artificial Sequence
  Framework 3 sequence of B7-H4 IgG1 (16E3H3 and 16E3H3 M1)
RVTMTRDTSISTAYMELSRLRSDDTAVYFCAR

SEQ ID NO: 15 - protein, 11 aa, Artificial Sequence
  Framework 4 sequence of B7-H4 IgG1 (16E3H3 and 16E3H3 M1)
WGAGTTVTVSS

SEQ ID NO: 16 - protein, 219 aa, Artificial Sequence
  Full sequence of Light Chain of B7-H4 IgG1 (16E3H3)
DVVMTQSPLSLPVTLGQPASISCRSSQSLVHSNGNTYLHWYLQRPGQSPRLLIYKVSNRFSGVPDRFSGSGSGTDFTLKISRVEAEDVGVYFCSQSTHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 23 aa, Artificial Sequence
  Framework 1 sequence of Light Chain of B7-H4 IgG1 (16E3H3)
DVVMTQSPLSLPVTLGQPASISC

SEQ ID NO: 18 - protein, 15 aa, Artificial Sequence
  Framework 2 sequence of Light Chain of B7-H4 IgG1 (16E3H3)
WYLQRPGQSPRLLIY

SEQ ID NO: 19 - protein, 32 aa, Artificial Sequence
  Framework 3 sequence of Light Chain of B7-H4 IgG1 (16E3H3)
GVPDRFSGSGSGTDFTLKISRVEAEDVGVYFC

SEQ ID NO: 20 - protein, 11 aa, Artificial Sequence
  Framework 4 sequence of Light Chain of B7-H4 IgG1 (16E3H3)
FGGGTKVEIKR

SEQ ID NO: 21 - protein, 219 aa, Artificial Sequence
  Full sequence of Light Chain of B7-H4 IgG1 (16E3H3 M1)
DVVMTQSPLSLPVTLGQPASISCRSSQSLVHSNANTYLHWYLQRPGQSPRLLIYKVSNRFSGVPDRFSGSGSGTDFTLKISRVEAEDVGVYFCSQSTHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 117 aa, Artificial Sequence
  Variable Heavy Chain sequence of 16E3H3 (from mouse)
QIQLVHSGPELKKPGETVKISCKASGYPFTTYGMNWVKQAPGKGLKWMGWIDTYSGVPTYADDFKGRFAFSLETSASTAYLQINNLKNEDTATYFCARRSFWYFDVWGTGTTVTVSS

SEQ ID NO: 23 - protein, 112 aa, Artificial Sequence
  Variable Light Chain sequence of 16E3H3 (from mouse)
DVVMTQTPLSLPVSLGDQASISCRSSQSLVHSNGNTYLHWYLQKPGQSPKLLIYKVSNRFSGVPDRFSGSGSGTDFTLKIRRVEAEDLGVYFCSQSTHVPWTFGGGTKLEIK

SEQ ID NO: 24 - protein, 121 aa, Artificial Sequence
  Variable Heavy Chain sequence of 23B6C2 (from mouse)
QVTLKESGPGILQPSQTLSLTCSFSGFSLSTFGMGVGWIRQPSGKGLEWLAHIWWDDDKYYNSALKSRLTISKETSKNQIFLKIANVDTTDTATYYCVRKDYYGYRGFAYWGQGTLVTVSA

SEQ ID NO: 25 - protein, 7 aa, Artificial Sequence
  HCDR1 sequence of 23B6C2
TFGMGVG

SEQ ID NO: 26 - protein, 16 aa, Artificial Sequence
  HCDR2 sequence of 23B6C2
HIWWDDDKYYNSALKS

SEQ ID NO: 27 - protein, 11 aa, Artificial Sequence
  HCDR3 sequence of 23B6C2
KDYYGYRGFAY

SEQ ID NO: 28 - protein, 115 aa, Artificial Sequence
  Variable Light Chain sequence of 23B6C2 (from mouse)
QLVLTQSSSASFSLGASAKLTCTLSSQHTTYTIEWYQQQPLKPPKYVMELKKDGSHSTGDGIPDRFSGSSSGADRYLCISNIQPEDEAIYICGVGDTIKEQFVYVFGGGTKVTVL

SEQ ID NO: 29 - protein, 12 aa, Artificial Sequence
  LCDR1 sequence of 23B6C2
TLSSQHTTYTIE

SEQ ID NO: 30 - protein, 11 aa, Artificial Sequence
  LCDR2 sequence of 23B6C2
LKKDGSHSTGD

SEQ ID NO: 31 - protein, 13 aa, Artificial Sequence
  LCDR3 sequence of 23B6C2
GVGDTIKEQFVYV

SEQ ID NO: 32 - protein, 120 aa, Artificial Sequence
  Variable Heavy Chain sequence of 73B4F10 (from mouse)
QVQLQQPGTELVKPGASVKLSCKASGYTFTSYWMHWVKQRPGQGLERIGHINPSNGGTNYNEKFKKKATLTVDKSSSTAYMQLSSLTSEDSAVYYCARSEFYYGTVFAYWGQGTLVTVSA

SEQ ID NO: 33 - protein, 5 aa, Artificial Sequence
  HCDR1 sequence of 73B4F10
SYWMH

SEQ ID NO: 34 - protein, 17 aa, Artificial Sequence
  HCDR2 sequence of 73B4F10
HINPSNGGTNYNEKFKK

SEQ ID NO: 35 - protein, 11 aa, Artificial Sequence
  HCDR3 sequence of 73B4F10
SEFYYGTVFAY

SEQ ID NO: 36 - protein, 112 aa, Artificial Sequence
  Variable Light Chain sequence of 73B4F10 (from mouse)
DVLMTQTPLSLPVSLGDQASISCRSSQSIVHGNGNTYLEWYLQKPGQSPKLLIYKVSNRFSGVPDRFSGSGSGTDFTLKISRVEAEDLGVYYCFQGSHDPPTFGGGTKLEIK

SEQ ID NO: 37 - protein, 16 aa, Artificial Sequence
  LCDR1 sequence of 73B4F10
RSSQSIVHGNGNTYLE

SEQ ID NO: 38 - protein, 9 aa, Artificial Sequence
  LCDR3 sequence of 73B4F10
FQGSHDPPT

SEQ ID NO: 39 - protein, 121 aa, Artificial Sequence
  Heavy Chain Variable Region sequence of 4-1BB scFv (1A10)
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDGQRNSMREFDYWGQGTLVTVSS

SEQ ID NO: 40 - protein, 121 aa, Artificial Sequence
  Heavy Chain Variable Region sequence of 4-1BB scFv (1A10M4,1A10M12)
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 41 - protein, 121 aa, Artificial Sequence
  Heavy Chain Variable Region sequence of 4-1BB scFv (1A10M11,1A10M13)
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRQSMREFDYWGQGTLVTVSS

SEQ ID NO: 42 - protein, 127 aa, Artificial Sequence
  Heavy Chain Variable Region sequence of 4-1BB scFv (1A12)
EVQLLESGGGLVQPGGSLRLSCAASGFTFSGYDMSWVRQAPGKCLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 43 - protein, 127 aa, Artificial Sequence
  Heavy Chain Variable Region sequence of 4-1BB scFv (1A12M1)
EVQLLESGGGLVQPGGSLRLSCAASGFTFSGYDMSWVRQAPGKCLEWVSVIYPDDGNTYYADSVKGRFTISRDNSKNTLYLQMNSLRAEDTAVYYCAKHGGQKPTTKSSSAYGMDGWGQGTLVTVSS

SEQ ID NO: 44 - protein, 5 aa, Artificial Sequence
  CDR H1 sequence of 4-1BB scFv (1A10, 1A10M4, 1A10M11, 1A10M12, 1A10M13)
SYDMS

SEQ ID NO: 45 - protein, 5 aa, Artificial Sequence
  CDR H1 sequence of 4-1BB scFv (1A12, 1A12M1)
GYDMS

SEQ ID NO: 46 - protein, 17 aa, Artificial Sequence
  CDR H2 sequence of 4-1BB scFv (1A10, 1A10M4, 1A10M11, 1A10M12, 1A10M13)
WISYSGGSIYYADSVKG

SEQ ID NO: 47 - protein, 17 aa, Artificial Sequence
  CDR H2 sequence of 4-1BB scFv (1A12, 1A12M1)
VIYPDDGNTYYADSVKG

SEQ ID NO: 48 - protein, 12 aa, Artificial Sequence
  CDR H3 sequence of 4-1BB scFv (1A10)
DGQRNSMREFDY

SEQ ID NO: 49 - protein, 12 aa, Artificial Sequence
  CDR H3 sequence of 4-1BB scFv (1A10M4, 1A10M12)
DAQRNSMREFDY

SEQ ID NO: 50 - protein, 12 aa, Artificial Sequence
  CDR H3 sequence of 4-1BB scFv (1A10M11, 1A10M13)
DAQRQSMREFDY

SEQ ID NO: 51 - protein, 18 aa, Artificial Sequence
  CDR H3 sequence of 4-1BB scFv (1A12, 1A12M1)
HGGQKPTTKSSSAYGMDG

SEQ ID NO: 52 - protein, 110 aa, Artificial Sequence
  Light chain variable regions sequence of 4-1BB scFv (1A10,1A10M4,1A10M11,1A12)
QSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 53 - protein, 110 aa, Artificial Sequence
  Light chain variable regions sequence of 4-1BB scFv (1A10M12,1A10M13,1A12M1)
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 54 - protein, 13 aa, Artificial Sequence
  CDR L1 sequence of 4-1BB scFv (1A10,1A10M4,1A10M11,1A10M12,1A10M13,1A12,1A12M1)
SGSSSNIGNNYVT

SEQ ID NO: 55 - protein, 7 aa, Artificial Sequence
  CDR L2 sequence of 4-1BB scFv (1A10,1A10M4,1A10M11,1A10M12,1A10M13,1A12,1A12M1)
ADSHRPS

SEQ ID NO: 56 - protein, 11 aa, Artificial Sequence
  CDR L3 sequence of 4-1BB scFv (1A10,1A10M4,1A10M11,1A10M12,1A10M13,1A12,1A12M1)
ATWDYSLSGYV

SEQ ID NO: 57 - protein, 30 aa, Artificial Sequence
  VH Framework 1 sequence of 4-1BB scFv (1A10,1A10M4,1A10M11,1A10M12,1A10M13,1A12,1A12M1)
EVQLLESGGGLVQPGGSLRLSCAASGFTFS

SEQ ID NO: 58 - protein, 14 aa, Artificial Sequence
  VH Framework 2 sequence of 4-1BB scFv (1A10,1A10M4,1A10M11,1A10M12,1A10M13,1A12,1A12M1)
WVRQAPGKCLEWVS

SEQ ID NO: 59 - protein, 32 aa, Artificial Sequence
  VH Framework 3 sequence of 4-1BB scFv (1A10,1A10M4,1A10M11,1A10M12,1A10M13)
RFTISRDNSKNTLYLQMNSLRAEDTAVYYCAR

SEQ ID NO: 60 - protein, 11 aa, Artificial Sequence
  VH Framework 4 sequence of 4-1BB scFv (1A10,1A10M4,1A10M11,1A10M12,1A10M13,1A12,1A12M1)
WGQGTLVTVSS

SEQ ID NO: 61 - protein, 22 aa, Artificial Sequence
  VL Framework 1 sequence of 4-1BB scFv (1A10,1A10M4,1A10M11,1A12)
QSVLTQPPSASGTPGRRVTISC

SEQ ID NO: 62 - protein, 15 aa, Artificial Sequence
  VL Framework 2 sequence of 4-1BB scFv (1A10,1A10M4,1A10M11,1A10M12,1A10M13,1A12,1A12M1)
WYQQLPGTAPKLLIY

SEQ ID NO: 63 - protein, 32 aa, Artificial Sequence
  VL Framework 3 sequence of 4-1BB scFv (1A10,1A10M4,1A10M11,1A10M12,1A10M13,1A12,1A12M1)
GVPDRFSGSKSGTSASLAISGLRSEDEADYYC

SEQ ID NO: 64 - protein, 10 aa, Artificial Sequence
  VL Framework 4 sequence of 4-1BB scFv (1A10,1A10M4,1A10M11,1A10M12,1A10M13,1A12,1A12M1)
FGCGTKLTVL

SEQ ID NO: 65 - protein, 22 aa, Artificial Sequence
  VL Framework 1 sequence of 4-1BB scFv (1A10M12,1A10M13,1A12M1)
QSVLTQPPSASGTPGQRVTISC

SEQ ID NO: 66 - protein, 32 aa, Artificial Sequence
  VL Framework 3 sequence of 4-1BB scFv (1A12)
RFTISRDNSKNTLYLQMNSLRAEDAAVYYCAK

SEQ ID NO: 67 - protein, 32 aa, Artificial Sequence
  VL Framework 3 sequence of 4-1BB scFv (1A12M1)
RFTISRDNSKNTLYLQMNSLRAEDTAVYYCAK

SEQ ID NO: 68 - protein, 20 aa, Artificial Sequence
  Linker between IgG1 and scFv, (GGGGS)4
GGGGSGGGGSGGGGSGGGGS

SEQ ID NO: 69 - protein, 18 aa, Artificial Sequence
  Linker between IgG1 and scFv, (GS)9
GSGSGSGSGSGSGSGSGS

SEQ ID NO: 70 - protein, 716 aa, Artificial Sequence
  Heavy chain sequence of 16E3H3 x1A10 M12
QVQLVQSGAEVKKPGASVKVSCKASGYPFTTYGMNWVKQAPGQGLEWMGWIDTYSGVPTYADDFKGRVTMTRDTSISTAYMELSRLRSDDTAVYFCARRSFWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 71 - protein, 716 aa, Artificial Sequence; identical to SEQ ID NO: 70
  Heavy chain sequence of 16E3H3 M1 x1A10 M12 (B10317)

SEQ ID NO: 72 - protein, 10 aa, Artificial Sequence
  the peptide linker
GGGGSGGGGS

SEQ ID NO: 73 - protein, 15 aa, Artificial Sequence
  the peptide linker
GGGGSGGGGSGGGGS

(19) United States
(12) Patent Application Publication
(10) Pub. No.: US 2024/0383983 A1
(43) Pub. Date: Nov. 21, 2024

(54) Anti-B7-H4/Anti-4-1BB Bispecific Antibodies And Use Thereof

(71) Applicant: ABL BIO, INC., Seongnam-Si (KR)

(72) Inventors: Kyungjin PARK, Seongnam-Si (KR); Yangsoon LEE, Seongnam-Si (KR); Saeyi LIM, Seongnam-Si (KR); Kyeongsu PARK, Seongnam-Si (KR); Minkyu SEON, Seongnam-Si (KR); Wonjun SON, Seongnam-Si (KR); Hyejin CHUNG, Seongnam-Si (KR); Yeryoung YONG, Seongnam-Si (KR); Yong-Gyu SON, Seongnam-Si (KR); Yeunju KIM, Seongnam-Si (KR); Youngdon PAK, Seongnam-Si (KR)

(21) Appl. No.: 18/684,660

(22) PCT Filed: Aug. 18, 2021
(86) PCT No.: PCT/KR2021/010945

§ 371 Date: Feb. 17, 2024

(30) Foreign Application Priority Data
Aug. 18, 2020 (KR) 10-2020-0103270

(51) Int. Cl. C07K 16/28; A61K 39/00; A61P 35/00

(52) U.S. Cl. CPC C07K 16/2827; A61P 35/00; C07K 16/2878; A61K 2039/505; C07K 2317/24; C07K 2317/31; C07K 2317/622; C07K 2317/732; C07K 2317/92

(57) ABSTRACT

The present disclosure provides an anti-B7-H4/anti-4-1BB bispecific antibody capable to effectively block the T-cell inhibitory effect of B7-H4 on the surface of tumor cells, and simultaneously activate 4-1BB on T-cells. The bispecific antibody may have high binding affinity to both of a B7-H4 protein (e.g., a human, monkey and mouse B7-H4 protein) and a 4-1BB protein (e.g., a human, monkey and mouse 4-1BB protein). The present disclosure also provides an anti-B7-H4 antibody specifically binds to B7-H4 protein.

### CROSS-REFERENCE TO RELATED APPLICATIONS

This application is a U.S. National Stage Entry of International Patent Application no. PCT/KR2021/010945, filed Aug. 18, 2021.

### REFERENCE TO AN ELECTRONIC SEQUENCE LISTING

This application contains a Sequence Listing which has been submitted electronically and is hereby incorporated by reference in its entirety. The Sequence Listing was created on Jan. 11, 2024, is named “24-0054-WO-US_SequenceListing_ST25” and is 53,857 bytes in size.

### TECHNICAL FIELD

The present disclosure relates to anti-B7-H4 antibodies, anti-B7-H4/anti-4-1BB bispecific antibodies and uses thereof.

### BACKGROUND ART

B7-H4 is a single-pass type I transmembrane protein and is a member of the B7 superfamily of proteins that provides co-signal in conjunction with a T-cell receptor antigenic signal. B7-H4 is a negative regulator of T-cell function and ligation of T-cells inhibits their growth, cytokine secretion and cytotoxicity.

Human B7-H4 is a 282 amino acid protein (including the amino-terminal signal sequence), of which 235 amino acids are predicted to be in the extracellular space following cleavage of the amino-terminal signal sequence. B7-H4 comprises an Ig-like V-domain, an Ig-like C domain, a transmembrane domain and a short cytoplasmic tail.

B7-H4 has the potential of down-regulating the immune system through its co-inhibitory signal in conjunction with antigen-dependent signaling by the T-cell receptor. B7-H4 is nominally expressed in normal human tissues but highly overexpressed in a myriad of human cancers including cancers of the female reproductive system—breast, ovarian, and endometrium. Prevalence of B7-H4 has been reported to be high in invasive ductal and lobular carcinomas comprising both primary (−95%) and metastatic breast cancer (−97%). Although increased B7-H4 staining was associated with negative PR and Her2 status, expression was independent of tumor grade or stage. In addition to the high proportion of B7H4 staining cells in those types of breast cancer, there was also a concomitant decrease in the number of infiltrating lymphocytes.

4-1BB is a member of TNF-receptor superfamily (TNFRSF) and is a co-stimulatory molecule which is expressed following the activation of immune cells, both innate and adaptive immune cells. 4-1BB plays important role in modulate the activity of various immune cells. 4-1BB agonists enhance immune cell proliferation, survival, secretion of cytokines and cytolytic activity CD8 T cells. Many other studies showed that activation of 4-1BB enhances immune response to eliminate tumors in mice. Therefore, it suggests that 4-1BB is a promising target molecule in cancer immunology. Despite of their anti-tumor efficacy, anti-4-1BB antibody induced severe liver toxicity in clinical application.

### DISCLOSURE

### Technical Problem

The technical problem to be solved by the present disclosure is to provide an anti-B7-H4/anti-4-1BB bispecific antibody or antigen-binding fragment thereof.

The technical problem to be solved by the present disclosure is to provide an anti-B7-H4 antibody or antigen-binding fragment thereof.

The technical problem to be solved by the present disclosure is to provide a pharmaceutical composition for prevention or treatment of a disease related to B7-H4, 4-1BB, or both thereof.

The technical problem to be solved by the present disclosure is to provide a pharmaceutical composition for prevention or treatment of a disease related to B7-H4.

The technical problem to be solved by the present disclosure is to provide a method of prevention or treatment of a disease related to B7-H4, 4-1BB, or both thereof in an individual.

The technical problem to be solved by the present disclosure is to provide a method of prevention or treatment of a disease related to B7-H4 thereof in an individual.

The technical problem to be solved by the present disclosure is to provide an anti-B7-H4/anti-4-1BB bispecific antibody or antigen-binding fragment thereof for use in the prevention or treatment of a disease related to B7-H4, 4-1BB, or both thereof.

The technical problem to be solved by the present disclosure is to provide an anti-B7-H4 antibody or antigen-binding fragment thereof for use in the prevention or treatment of a disease related to B7-H4.

### Technical Solution

### Definitions

As used herein, the term “a” or “an” entity may refer to one or more of that entity, for example, “an antibody,” is understood to represent one or more antibodies. As such, the terms “a” (or “an”), “one or more,” and “at least one” can be used interchangeably herein.

As used herein, the term ‘consisting of a sequence,’ ‘consisting essentially of a sequence,’ or ‘comprising a sequence’ may refer to any case comprising the sequence, but it may not be intended to exclude a case comprising further sequence other than the sequence.

As used herein, the term ‘a protein or polypeptide comprising or consisting of an amino acid sequence identified by SEQ ID NO’ and ‘a gene or polynucleotide comprising or consisting of a nucleic acid sequence identified by SEQ ID NO’ may refer to a protein (or polypeptide) or gene (or polynucleotide), which consists essentially of the amino acid sequence or nucleic acid sequence, or which has at least 90%, at least 91%, at least 92%, at least 93%, at least 94%, at least 95%, at least 96%, at least 97%, at least 98%, or at least 99% sequence identity with the amino acid sequence or nucleic acid sequence with maintaining its inherent activity and/or function.

As used herein, the term “antibody” may encompass various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4), and light chains are classified as either kappa or lambda (K, λ). It is the nature of this chain that determines the “class” of the antibody as IgG, IgM, IgA IgG, or IgE, respectively. The immunoglobulin subclasses (isotypes), e.g., IgG1, IgG2, IgG3, IgG4, IgG5, etc., are well characterized and are known to confer functional specialization.

The term “heavy chain” refers to a full-length heavy chain or a fragment thereof, including a variable region VH that includes amino acid sequences sufficient to provide specificity to antigens, and three constant regions, CH1, CH2, and CH3, and a hinge. The term “light chain” refers to a full-length light chain or a fragment thereof, including a variable region VL that includes amino acid sequences sufficient to provide specificity to antigens, and a constant region CL.

The term “complementarity determining region (CDR)” refers to an amino acid sequence found in a hyper variable region of a heavy chain or a light chain of immunoglobulin. The heavy and light chains may respectively include three CDRs (CDRH1, CDRH2, and CDRH3; and CDRL1, CDRL2, and CDRL3). The CDR may provide residues that play an important role in the binding of antibodies to an antigens or epitope. The terms “specifically binding” or “specifically recognized” is well known to one of ordinary skill in the art, and indicates that an antibody and an antigen specifically interact with each other to lead to an immunological activity.

In this disclosure, the antibody may include, but not be limited to, polyclonal or monoclonal; and/or human, humanized, animal (e.g., mouse, rabbit, etc.) derived antibody, or chimeric antibodies (e.g., mouse-human chimeric antibody). In addition, the antibody of the present disclosure may include monospecific antibody and multispecific antibody such as bispecific antibody and trispecific antibody, but it may not be limited thereto.

As used herein, the term “antigen binding fragment” refers to a fragment derived from a full immunoglobulin structure comprising a portion capable of binding to an antigen such as CDRs. For example, the antigen binding fragment may be or may include scFv, (scFv)₂, Fab, Fab′, F(ab′)₂, or any combination thereof but not be limited thereto. In the present disclosure, the antigen binding fragment may be a fragment derived from an antibody, comprising at least one complementarity determining region, for example, selected from the group consisting of scFv, (scFv)₂, scFv-Fc, Fab, Fab′ and F(ab′)₂.

For example, the antibody or antigen binding fragment may be chemically or recombinantly synthesized (not naturally occurring).

### Anti-B7-H4 Antibody

The anti-B7-H4/anti-4-1BB bispecific antibody may comprise an anti-B7-H4 antibody or an antigen-binding fragment thereof as a B7-H4 targeting moiety.

In an embodiment, the anti-B7-H4 antibody or fragment thereof can specifically bind to B7-H4 (e.g., human B7-H4) protein.

As a member of the B7 family, B7-H4 (also known as V-set Domain-Containing T Cell Activation Inhibitor 1 (VTCN1)) is involved in the control of T cell activation. B7-H4 negatively regulates T cell immune response by inhibiting the proliferation cytokine secretion and cell cycle of T cells. Viewed from a structural perspective, B7-H4 is a member of the immunoglobulin (Ig) superfamily containing two Ig-like domains: a variable (IgV) and a constant (IgC) domain. Similar to another negative co-stimulators, PD-L1, B7-H4 is expressed not only by APCs (antigen presenting cells) but also in a variety of normal non-lymphoid and cancerous tissues. The expression by non-APCs implicates that B7-H4 might also have additional functions to the one of a classical co-stimulatory molecule.

The human protein of B7-H4 consists of the amino acid sequence is NCBI Reference Sequence ID: NP_001240778.1, NP_001240779.1, or NP_078902.2; and the nucleic acid sequence is NM_001253849.2, NM_001253850.2, or NM_024626.4. Unless apparent from the context used herein, the B7-H4 refers to a human B7-H4, but the antibody also has the binding capacity to monkey B7-H4. The mouse B7-H4 amino acid sequence is represented by GenBank: XP_001103715.

Tissue specific overexpression of B7-H4 was correlated with negative clinical outcomes in prostate cancer, ovarian cancer, breast cancer, pancreatic cancer, and renal cancer. Recently, the inventors showed that impaired B7-H4 presentation marks type I diabetes development in both mouse models and human patients, linking this process to a proteolytic shedding of cell surface B7-H4 from APCs and pancreatic islets. Ig treatment reduced the incidence of T1D, experimental autoimmune encephalomyelitis, and rheumatoid arthritis, highlighting its importance as a potential therapeutic target for both autoimmunity and cancer.

The anti-B7-H4 antibody or antigen-binding fragment thereof may exhibit potent binding activities to B7-H4, and be useful for therapeutic and/or diagnostics uses.

In an embodiment, an anti-B7-H4/anti-4-1BB bispecific antibody, comprising an anti-B7-H4 antibody or an antigen-binding fragment thereof and an anti-4-1BB antibody or an antigen-binding fragment thereof, wherein the anti-B7-H4 antibody or an antigen-binding fragment thereof includes a heavy chain variable region and a light chain variable region comprising:
a heavy chain complementarity-determining region 1 (CDR-H1) comprising an amino acid sequence selected from the group consisting of SEQ ID NOs: 2, 25 and 33;
a CDR-H2 comprising an amino acid sequence selected from the group consisting of SEQ ID NOs: 3, 26 and 34;
a CDR-H3 comprising an amino acid sequence selected from the group consisting of SEQ ID NOs: 4, 27 and 35;
a light chain complementarity-determining region 1 (CDR-L1) comprising an amino acid sequence selected from the group consisting of SEQ ID NOs: 7, 8, 29 and 37;
a CDR-L2 comprising an amino acid sequence selected from the group consisting of SEQ ID NOs: 9 and 30; and
a CDR-L3 comprising an amino acid sequence selected from the group consisting of SEQ ID NOs: 10, 31 and 38;
wherein the anti-4-1BB antibody or an antigen-binding fragment thereof comprises a heavy chain variable region comprising at least one amino acid sequence selected from the group consisting of SEQ ID NOs: 39 to 43; and a light chain variable region comprising at least one amino acid sequence selected from the group consisting of SEQ ID NOs: 52 and 53. The amino acid sequences of the Heavy chain CDRs of the anti-B7-H4 antibody or an antigen-binding fragment thereof are listed in Table 1.

TABLE 1
Examples of Heavy chain CDRs of anti-B7-H4 antibody or an antigen-
binding fragment thereof according to Kabat definition

| | | SEQ ID | | SEQ ID | | SEQ ID |
|---|---|---|---|---|---|---|
| Antibody | CDR H1 | NO | CDR H2 | NO | H3 CDR | NO |
| 16E3H3 | TYGMN | 2 | WIDTYSGVPTY | 3 | RSFWYFDV | 4 |
| | | | ADDFKG | | | |
| 16E3H3 | TYGMN | 2 | WIDTYSGVPTY | 3 | RSFWYFDV | 4 |
| M1 | | | ADDFKG | | | |
| 23B6C2 | TFGMGVG | 25 | HIWWDDDKY | 26 | KDYYGYRGF | 27 |
| | | | YNSALKS | | AY | |
| 73B4F10 | SYWMH | 33 | HINPSNGGTN | 34 | SEFYYGTVFA | 35 |
| | | | YNEKFKK | | Y | |

The amino acid sequences of the Heavy chain variable regions of the anti-B7-H4 antibody or an antigen-binding fragment thereof are listed in Table 2.

TABLE 2
Examples of Heavy chain variable regions of anti-B7-H4 antibody or an
antigen-binding fragment thereof according to Kabat definition

| Antibody | VH Amino acid Sequence | SEQ ID NO |
|---|---|---|
| 16E3H3 | QVQLVQSGAEVKKPGASVKVSCKASGYPFTTYGMN | 1 |
| (humanized) | WVKQAPGQGLEWMGWIDTYSGVPTYADDFKGRVT | |
| | MTRDTSISTAYMELSRLRSDDTAVYFCARRSFWYFDV | |
| | WGAGTTVTVSS | |
| 16E3H3 M1 | QVQLVQSGAEVKKPGASVKVSCKASGYPFTTYGMN | 1 |
| (humanized) | WVKQAPGQGLEWMGWIDTYSGVPTYADDFKGRVT | |
| | MTRDTSISTAYMELSRLRSDDTAVYFCARRSFWYFDV | |
| | WGAGTTVTVSS | |
| 16E3H3 | QIQLVHSGPELKKPGETVKISCKASGYPFTTYGMNW | 22 |
| (from mouse) | VKQAPGKGLKWMGWIDTYSGVPTYADDFKGRFAFS | |
| | LETSASTAYLQINNLKNEDTATYFCARRSFWYFDVWG | |
| | TGTTVTVSS | |
| 23B6C2 | QVTLKESGPGILQPSQTLSLTCSFSGFSLSTFGMGVG | 24 |
| (from mouse) | WIRQPSGKGLEWLAHIWWDDDKYYNSALKSRLTISK | |
| | ETSKNQIFLKIANVDTTDTATYYCVRKDYYGYRGFAY | |
| | WGQGTLVTVSA | |
| 73B4F10 | QVQLQQPGTELVKPGASVKLSCKASGYTFTSYWMH | 32 |
| (from mouse) | WVKQRPGQGLERIGHINPSNGGTNYNEKFKKKATLT | |
| | VDKSSSTAYMQLSSLTSEDSAVYYCARSEFYYGTVFAY | |
| | WGQGTLVTVSA | |

The amino acid sequences of the Light chain CDRs of the anti-17-1H4 antibody or an antigen-binding fragment thereof are listed in Table 3.

TABLE 3
Examples of Light chain variable CDRs of anti-B7-H4 antibody or an
antigen-binding fragment thereof according to Kabat definition

| | | SEQ ID | | SEQ ID | | SEQ ID |
|---|---|---|---|---|---|---|
| Antibody | CDR L1 | NO | CDR L2 | NO | L3 CDR | NO |
| 16E3H3 | RSSQSLVHSN | 7 | KVSNRFS | 9 | SQSTHVPWT | 10 |
| | GNTYLH | | | | | |
| 16E3H3 | RSSQSLVHSN | 8 | KVSNRFS | 9 | SQSTHVPWT | 10 |
| M1 | ANTYLH | | | | | |
| 23B6C2 | TLSSQHTTYTIE | 29 | LKKDGSHSTGD | 30 | GVGDTIKEQFV | 31 |
| | | | | | YV | |
| 73B4F10 | RSSQSIVHGN | 37 | KVSNRFS | 9 | FQGSHDPPT | 38 |
| | GNTYLE | | | | | |

The amino acid sequences of the Light chain variable regions of the anti-B7-H4 antibody or an antigen-binding fragment thereof are listed in Table 4.

TABLE 4
Examples of Light chain variable regions of anti-B7-H4 antibody or an
antigen-binding fragment thereof according to Kabat definition

| Antibody | VL Amino acid Sequence | SEQ ID NO |
|---|---|---|
| 16E3H3 | DVVMTQSPLSLPVTLGQPASISCRSSQSLVHSNGNT | 5 |
| (humanized) | YLHWYLQRPGQSPRLLIYKVSNRFSGVPDRFSGSGS | |
| | GTDFTLKISRVEAEDVGVYFCSQSTHVPWTFGGGTK | |
| | VEIKR | |
| 16E3H3 M1 | DVVMTQSPLSLPVTLGQPASISCRSSQSLVHSNANT | 6 |
| (humanized) | YLHWYLQRPGQSPRLLIYKVSNRFSGVPDRFSGSGS | |
| | GTDFTLKISRVEAEDVGVYFCSQSTHVPWTFGGGTK | |
| | VEIKR | |
| 16E3H3 | DVVMTQTPLSLPVSLGDQASISCRSSQSLVHSNGNT | 23 |
| (from mouse) | YLHWYLQKPGQSPKLLIYKVSNRFSGVPDRFSGSGS | |
| | GTDFTLKIRRVEAEDLGVYFCSQSTHVPWTFGGGTKL | |
| | EIK | |
| 23B6C2 | QLVLTQSSSASFSLGASAKLTCTLSSQHTTYTIEWYQ | 28 |
| (from mouse) | QQPLKPPKYVMELKKDGSHSTGDGIPDRFSGSSSGA | |
| | DRYLCISNIQPEDEAIYICGVGDTIKEQFVYVFGGGTK | |
| | VTVL | |
| 73B4F10 | DVLMTQTPLSLPVSLGDQASISCRSSQSIVHGNGNT | 36 |
| (from mouse) | YLEWYLQKPGQSPKLLIYKVSNRFSGVPDRFSGSGSG | |
| | TDFTLKISRVEAEDLGVYYCFQGSHDPPTFGGGTKLEI | |
| | K | |

In one embodiment, the anti-17-1H4 antibody or an antigen binding fragment thereof can be designed by suitably mixing and matching the CDRs listed in Table 1 and 3 so as to maintain its affinity to 1B7-1H4. For example, the anti-17-1H4 antibody or an antigen binding fragment thereof may comprise:
(1) a CDR-H1 of SEQ ID NO: 2, a CDR-H-2 of SEQ ID NO: 3, a CDR-H-3 of SEQ ID NO: 4, a CDR-L1 of SEQ ID NO: 7, a CDR-L2 of SEQ ID NO: 9, and a CDR-L3 of SEQ ID NO: 10;
(2) a CDR-H1 of SEQ ID NO: 2, a CDR-H2 of SEQ ID NO: 3, a CDR-H3 of SEQ ID NO: 4, a CDR-L1 of SEQ ID NO: 8, a CDR-L2 of SEQ ID NO: 9, and a CDR-L3 of SEQ ID NO: 10;
(3) a CDR-H1 of SEQ ID NO: 25, a CDR-H2 of SEQ ID NO: 26, a CDR-H3 of SEQ ID NO: 27, a CDR-L1 of SEQ ID NO: 29, a CDR-L2 of SEQ ID NO: 30, and a CDR-L3 of SEQ ID NO: 31; or
(4) a CDR-H1 of SEQ ID NO: 33, a CDR-H2 of SEQ ID NO: 34, a CDR-H3 of SEQ ID NO: 35, a CDR-L1 of SEQ ID NO: 37, a CDR-L2 of SEQ ID NO: 9, and a CDR-L3 of SEQ ID NO: 38.

In one embodiment, the anti-B7-H4 antibody or an antigen binding fragment thereof comprises:
a heavy chain variable region comprising an amino acid sequence selected from the group consisting of SEQ ID NO: 1, 22, 24 and 32; and
a light chain variable region comprising an amino acid selected from the group consisting of SEQ ID NOs: 5, 6, 23, 28 and 36.

Examples of heavy chain variable regions and light chain variable regions of the antibody or antigen-binding fragment are listed in the Table 2 and 4.

In other embodiment, the variable regions of heavy chain and light chain disclosed in Table 2 and 4, can be suitably combined (mixed and marched) for preparation of various forms of antibodies, and for example, they can form a single-chain antibody such as ScFV, or domain antibody, or full-length antibody (e.g., an IgG form antibody comprising two heavy chains and two light chains).

For example, the anti-B7-H4 antibody or antigen-binding fragment may comprise:
(1) a heavy chain variable region of SEQ ID NO: 1 and a light chain variable region of SEQ ID NO: 5;
(2) a heavy chain variable region of SEQ ID NO: 1 and a light chain variable region of SEQ ID NO: 6;
(3) a heavy chain variable region of SEQ ID NO: 22 and a light chain variable region of SEQ ID NO: 23;
(4) a heavy chain variable region of SEQ ID NO: 24 and a light chain variable region of SEQ ID NO: 28; or
(5) a heavy chain variable region of SEQ ID NO: 32 and a light chain variable region of SEQ ID NO: 36.

Each of heavy chain variable regions and light chain variable regions disclosed herein may be combined with various constant regions of heavy chain and light chain to form heavy chain and light chain of an intact antibody, respectively.

Non-limiting examples of the anti-B7-H4 antibody or an antigen binding fragment thereof may comprise:
a heavy chain variable region comprising an amino acid sequence selected from the group consisting of SEQ ID NO: 1, 22, 24 and 32, or an amino acid sequence having a sequence identity of at least 80%, at least 85%, at least 90%, at least 95%, at least 96%, at least 97%, at least 98%, or at least 99% to the above-described amino acid sequences; and
a light chain variable region comprising an amino acid sequence selected from SEQ ID NOs: 5, 6, 23, 28 and 36, or an amino acid sequence having a sequence identity of at least 80%, at least 85%, at least 90%, at least 95%, at least 96%, at least 97%, at least 98%, or at least 99% to the above-described amino acid sequences.

In one embodiment, the anti-B7-H4 antibody or an antigen binding fragment thereof comprises: a heavy chain framework 1 (H-FR1) comprising an amino acid sequence of SEQ ID NO: 12; a heavy chain framework 2 (H-FR2) comprising an amino acid sequence of SEQ ID NO: 13; a heavy chain framework 3 (H-FR3) comprising an amino acid sequence of SEQ ID NO: 14; a heavy chain framework 4 (H-FR4) comprising an amino acid sequence of SEQ ID NO: 15; a light chain framework 1 (L-FR1) comprising an amino acid sequence of SEQ ID NO: 17; a light chain framework 2 (L-FR2) comprising an amino acid sequence of SEQ ID NO: 18; a light chain framework 3 (L-FR3) comprising an amino acid sequence of SEQ ID NO: 19; and a light chain framework 4 (L-FR4) comprising an amino acid sequence of SEQ ID NO: 20.

In an embodiment, if the anti-B7-H4 antibody or an antigen binding fragment is a form of scFv, the heavy chain variable region and the light chain variable region may be linked via a suitable peptide linker. For example, the peptide linker may be (GGGGS)2 which is SEQ ID NO: 72, (GGGGS)3 which is SEQ ID NO: 73, (GGGGS)4 which is SEQ ID NO: 68, or (GS)9 which is SEQ ID NO: 69.

For example, binding affinities of an anti-B7-H4 antibody or an antigen binding fragment thereof of the disclosure to human B7-H4 include those with a dissociation constant or KD of 1×10⁻⁶ M or less, 1×10⁻⁷ M or less, 1×10⁻⁸ M or less, 1×10⁻⁹ M or less, or 1×10⁻¹⁰ M or less.

### Anti-4-1BB Antibody

The anti-B7-H4/anti-4-1BB bispecific antibody may comprise an anti-4-1BB antibody or an antigen-binding fragment thereof as a 4-1BB targeting moiety. WO2020/111913, which discloses anti-4-1BB antibodies, is incorporated herein by reference in its entirety.

In an embodiment, the anti-4-1BB antibody or fragment thereof can specifically bind to 4-1BB (e.g., human 4-1BB) protein.

For example, the human 4-1BB protein may be selected from the group consisting of proteins represented by NCBI Accession No. NP_001552.2, etc., but may not be limited thereto. These anti-4-1BB antibodies or antigen-binding fragments thereof are capable of enhancing immune response and/or treating tumor (cancer) in a mammal. The anti-4-1BB antibody or an antigen-binding fragment thereof is characterized by being localized and/or activated in tumor microenvironment (TME) and/or considerably reducing liver toxicities compared to pre-existing anti-4-1BB antibodies, with maintaining the efficacies of enhancing immune response enhancement and/or tumor treatment.

The term “4-1-BB” refers to CD137, or TNFRSF9 (TNF Receptor 25 Superfamily Member 9), is a member of TNF-receptor superfamily (TNFRSF) and is a co-stimulatory molecule which is expressed following the activation of immune cells, both innate and adaptive immune cells. As used herein, 4-1BB may be originated from a mammal, for example, Homo sapiens (human) (NCBI Accession No. NP_001552.2).

As described herein, the term “4-1BB” includes variants, isoforms, homologs, orthologs, and paralogs. For example, antibodies specific for a human 4-1BB protein may, in certain cases, cross-react with a 4-1BB protein from a species other than human. In other embodiments, the antibodies specific for a human 4-1BB protein may be completely specific for the human 4-1BB protein and may exhibit species or other types of cross-reactivity, or may cross-react with 4-1BB from certain other species but not all other species (e.g., cross-react with monkey 4-1BB, but not mouse 4-1BB). The term “human 4-1BB” refers to human sequence 4-1BB, such as the complete amino acid sequence of human 4-1BB having NCBI Accession No. NP_001552.2. The term “mouse 4-1BB” refers to mouse sequence 4-1BB, such as the complete amino acid sequence of mouse 4-1BB having NCBI Accession No. NP 033430.1. 4-1BB also can be known in the art as, for example, CD137. The human 4-1BB sequence in the disclosure may differ from human 4-1BB of NCBI Accession No. NP_001552.2 by having, e.g., conserved mutations or mutations in non-conserved regions and the 4-1BB in the disclosure has substantially the same biological function as the human 4-1BB of NCBI Accession No. NP_001552.2.

In an embodiment, the anti-4-1BB antibody or antigen-binding fragment thereof may comprise:
a CDR-H1 comprising an amino acid sequence selected from SEQ ID NOs: 44 and 45;
a CDR-H2 comprising an amino acid sequence selected from SEQ ID NOs: 46 and 47; and
a CDR-H3 comprising an amino acid sequence selected from SEQ ID NOs: 48 to 51;
a CDR-L1 comprising an amino acid sequence of SEQ ID NO: 54;
a CDR-L2 comprising an amino acid sequence of SEQ ID NO: 55; and
a CDR-L3 comprising an amino acid sequence of SEQ ID NO: 56.

The amino acid sequences of the Heavy chain CDRs of the anti-4-1BB antibody or an antigen-binding fragment thereof are listed in Table 5.

TABLE 5
Examples of Heavy chain CDRs of anti-4-1BB antibody or an antigen-
binding fragment thereof according to Kabat definition

| | | SEQ ID | | SEQ ID | | SEQ ID |
|---|---|---|---|---|---|---|
| Antibody | CDR H1 | NO | CDR H2 | NO | CDR H3 | NO |
| 1A10 | SYDMS | 44 | WISYSGGSIYY | 46 | DGQRNSMR | 48 |
| | | | ADSVKG | | EFDY | |
| 1A10M4 | SYDMS | 44 | WISYSGGSIYY | 46 | DAQRNSMR | 49 |
| | | | ADSVKG | | EFDY | |
| 1A10M11 | SYDMS | 44 | WISYSGGSIYY | 46 | DAQRQSMR | 50 |
| | | | ADSVKG | | EFDY | |
| 1A10M12 | SYDMS | 44 | WISYSGGSIYY | 46 | DAQRNSMR | 49 |
| | | | ADSVKG | | EFDY | |
| 1A10M13 | SYDMS | 44 | WISYSGGSIYY | 46 | DAQRQSMR | 50 |
| | | | ADSVKG | | EFDY | |
| 1A12 | GYDMS | 45 | VIYPDDGNTYY | 47 | HGGQKPTTK | 51 |
| | | | ADSVKG | | SSSAYGMDG | |
| 1A12M1 | GYDMS | 45 | VIYPDDGNTYY | 47 | HGGQKPTTK | 51 |
| | | | ADSVKG | | SSSAYGMDG | |

The amino acid sequences of the Heavy chain variable regions of the anti-4-1BB antibody or an antigen-binding fragment thereof are listed in Table 6.

TABLE 6
Examples of Heavy chain variable regions of anti-4-1BB antibody or an
antigen-binding fragment thereof according to Kabat definition

| Antibody | VH Amino acid Sequence | SEQ ID NO |
|---|---|---|
| 1A10 | EVQLLESGGGLVQPGGSLRLSCAASGFTFSSYDMSWV | 39 |
| | RQAPGKCLEWVSWISYSGGSIYYADSVKGRFTISRDN | |
| | SKNTLYLQMNSLRAEDTAVYYCARDGQRNSMREFDY | |
| | WGQGTLVTVSS | |
| 1A10M4 | EVQLLESGGGLVQPGGSLRLSCAASGFTFSSYDMSWV | 40 |
| | RQAPGKCLEWVSWISYSGGSIYYADSVKGRFTISRDN | |
| | SKNTLYLQMNSLRAEDTAVYYCARDAQRNSMREFDY | |
| | WGQGTLVTVSS | |
| 1A10M11 | EVQLLESGGGLVQPGGSLRLSCAASGFTFSSYDMSWV | 41 |
| | RQAPGKCLEWVSWISYSGGSIYADSVKGRFTISRDN | |
| | SKNTLYLQMNSLRAEDTAVYYCARDAQRQSMREFDY | |
| | WGQGTLVTVSS | |
| 1A10M12 | EVQLLESGGGLVQPGGSLRLSCAASGFTFSSYDMSWV | 40 |
| | RQAPGKCLEWVSWISYSGGSIYYADSVKGRFTISRDN | |
| | SKNTLYLQMNSLRAEDTAVYYCARDAQRNSMREFDY | |
| | WGQGTLVTVSS | |
| 1A10M13 | EVQLLESGGGLVQPGGSLRLSCAASGFTFSSYDMSWV | 41 |
| | RQAPGKCLEWVSWISYSGGSIYYADSVKGRFTISRDN | |
| | SKNTLYLQMNSLRAEDTAVYYCARDAQRQSMREFDY | |
| | WGQGTLVTVSS | |
| 1A12 | EVQLLESGGGLVQPGGSLRLSCAASGFTFSGYDMSW | 42 |
| | VRQAPGKCLEWVSVIYPDDGNTYYADSVKGRFTISRD | |
| | NSKNTLYLQMNSLRAEDAAVYYCAKHGGQKPTTKSS | |
| | SAYGMDGWGQGTLVTVSS | |
| 1A12M1 | EVQLLESGGGLVQPGGSLRLSCAASGFTFSGYDMSW | 43 |
| | VRQAPGKCLEWVSVIYPDDGNTYYADSVKGRFTISRD | |
| | NSKNTLYLQMNSLRAEDTAVYYCAKHGGQKPTTKSSS | |
| | AYGMDGWGQGTLVTVSS | |

The amino acid sequences of the Light chain CDRs of the anti-4-1BB antibody or an antigen-binding fragment thereof are listed in Table 7.

TABLE 7
Examples of Light chain CDRs of anti-4-1BB antibody or an antigen-
binding fragment thereof according to Kabat definition

| | | SEQ ID | | SEQ ID | | SEQ ID |
|---|---|---|---|---|---|---|
| Antibody | CDR L1 | NO | CDR L2 | NO | CDR L3 | NO |
| 1A10 | SGSSSNIGNN | 54 | ADSHRPS | 55 | ATWDYSLSGYV | 56 |
| | YVT | | | | | |
| 1A10M4 | SGSSSNIGNN | 54 | ADSHRPS | 55 | ATWDYSLSGYV | 56 |
| | YVT | | | | | |
| 1A10M11 | SGSSSNIGNN | 54 | ADSHRPS | 55 | ATWDYSLSGYV | 56 |
| | YVT | | | | | |
| 1A10M12 | SGSSSNIGNN | 54 | ADSHRPS | 55 | ATWDYSLSGYV | 56 |
| | YVT | | | | | |
| 1A10M13 | SGSSSNIGNN | 54 | ADSHRPS | 55 | ATWDYSLSGYV | 56 |
| | YVT | | | | | |
| 1A12 | SGSSSNIGNN | 54 | ADSHRPS | 55 | ATWDYSLSGYV | 56 |
| | YVT | | | | | |
| 1A12M1 | SGSSSNIGNN | 54 | ADSHRPS | 55 | ATWDYSLSGYV | 56 |
| | YVT | | | | | |

The amino acid sequences of the Light chain variable regions of the anti-4-1BB antibody or an antigen-binding fragment thereof are listed in Table 8.

TABLE 8
Examples of Light chain variable regions of anti-4-1BB antibody or an
antigen-binding fragment thereof according to Kabat definition

| Antibody | VL Amino acid Sequence | SEQ ID NO |
|---|---|---|
| 1A10 | QSVLTQPPSASGTPGRRVTISCSGSSSNIGNNYVTWY | 52 |
| | QQLPGTAPKLLIYADSHRPSGVPDRFSGSKSGTSASL | |
| | AISGLRSEDEADYYCATWDYSLSGYVFGCGTKLTVL | |
| 1A10M4 | QSVLTQPPSASGTPGRRVTISCSGSSSNIGNNYVTWY | 52 |
| | QQLPGTAPKLLIYADSHRPSGVPDRFSGSKSGTSASL | |
| | AISGLRSEDEADYYCATWDYSLSGYVFGCGTKLTVL | |
| 1A10M11 | QSVLTQPPSASGTPGRRVTISCSGSSSNIGNNYVTWY | 52 |
| | QQLPGTAPKLLIYADSHRPSGVPDRFSGSKSGTSASL | |
| | AISGLRSEDEADYYCATWDYSLSGYVFGCGTKLTVL | |
| 1A10M12 | QSVLTQPPSASGTPGQRVTISCSGSSSNIGNNYVTW | 53 |
| | YQQLPGTAPKLLIYADSHRPSGVPDRFSGSKSGTSASL | |
| | AISGLRSEDEADYYCATWDYSLSGYVFGCGTKLTVL | |
| 1A10M13 | QSVLTQPPSASGTPGQRVTISCSGSSSNIGNNYVTW | 53 |
| | YQQLPGTAPKLLIYADSHRPSGVPDRFSGSKSGTSASL | |
| | AISGLRSEDEADYYCATWDYSLSGYVFGCGTKLTVL | |
| 1A12 | QSVLTQPPSASGTPGRRVTISCSGSSSNIGNNYVTWY | 52 |
| | QQLPGTAPKLLIYADSHRPSGVPDRFSGSKSGTSASL | |
| | AISGLRSEDEADYYCATWDYSLSGYVFGCGTKLTVL | |
| 1A12M1 | QSVLTQPPSASGTPGQRVTISCSGSSSNIGNNYVTW | 53 |
| | YQQLPGTAPKLLIYADSHRPSGVPDRFSGSKSGTSASL | |
| | AISGLRSEDEADYYCATWDYSLSGYVFGCGTKLTVL | |

In one embodiment, the anti-4-1BB antibody or an antigen binding fragment thereof can be designed by suitably mixing and matching the CDRs listed in Table 5 and 7 so as to maintain its affinity to 4-1BB. For example, the anti-4-1BB antibody or an antigen binding fragment thereof may comprise:
(1) a CDR-H1 of SEQ ID NO: 44, a CDR-H2 of SEQ ID NO: 46, a CDR-H3 of SEQ ID NO: 48, a CDR-L1 of SEQ ID NO: 54, a CDR-L2 of SEQ ID NO: 55, and a CDR-L3 of SEQ ID NO: 56;
(2) a CDR-H1 of SEQ ID NO: 44, a CDR-H2 of SEQ ID NO: 46, a CDR-H3 of SEQ ID NO: 49, a CDR-L1 of SEQ ID NO: 54, a CDR-L2 of SEQ ID NO: 55, and a CDR-L3 of SEQ ID NO: 56;
(3) a CDR-H1 of SEQ ID NO: 44, a CDR-H2 of SEQ ID NO: 46, a CDR-H3 of SEQ ID NO: 50, a CDR-L1 of SEQ ID NO: 54, a CDR-L2 of SEQ ID NO: 55, and a CDR-L3 of SEQ ID NO: 56;
(4) a CDR-H1 of SEQ ID NO: 44, a CDR-H2 of SEQ ID NO: 46, a CDR-H3 of SEQ ID NO: 49, a CDR-L1 of SEQ ID NO: 54, a CDR-L2 of SEQ ID NO: 55, and a CDR-L3 of SEQ ID NO: 56;
or
(5) a CDR-H1 of SEQ ID NO: 45, a CDR-H2 of SEQ ID NO: 47, a CDR-H3 of SEQ ID NO: 51,
a CDR-L1 of SEQ ID NO: 54, a CDR-L2 of SEQ ID NO: 55, and a CDR-L3 of SEQ ID NO: 56.

Examples of heavy chain variable regions and light chain variable regions of the antibody or antigen-binding fragment are listed in the Table 6 and 8.

Non-limiting examples of the anti-4-1BB antibody or an antigen binding fragment thereof may comprise:
a heavy chain variable region comprising an amino acid sequence of selected from the group consisting of SEQ ID NOs: 39 to 43 or an amino acid sequence having a sequence identity of at least 80%, at least 85%, at least 90%, at least 95%, at least 96%, at least 97%, at least 98%, or at least 99% to the above described amino acid sequences; and
a light chain variable region comprising an amino acid sequence selected from SEQ ID NOs: 52 and 53, or an amino acid sequence having a sequence identity of at least 80%, at least 85%, at least 90%, at least 95%, at least 96%, at least 97%, at least 98%, or at least 99% to the above described amino acid sequences.

In other embodiment, the variable regions of heavy chain and light chain disclosed in Table 6 and 8, can be suitably combined (mixed and marched) for preparation of various forms of antibodies, and for example, they can form a single-chain antibody such as scFV, or domain antibody, or full-length antibody (e.g., an IgG form antibody comprising two heavy chains and two light chains).

For example, the anti-4-1BB antibody or antigen-binding fragment may comprise:
(1) a heavy chain variable region of SEQ ID NO: 39 and a light chain variable region of SEQ ID NO: 52;
(2) a heavy chain variable region of SEQ ID NO: 40 and a light chain variable region of SEQ ID NO: 52;
(3) a heavy chain variable region of SEQ ID NO: 41 and a light chain variable region of SEQ ID NO: 52;
(4) a heavy chain variable region of SEQ ID NO: 40 and a light chain variable region of SEQ ID NO: 53;
(5) a heavy chain variable region of SEQ ID NO: 41 and a light chain variable region of SEQ ID NO: 53;
(6) a heavy chain variable region of SEQ ID NO: 42 and a light chain variable region of SEQ ID NO: 52;
(7) a heavy chain variable region of SEQ ID NO: 43 and a light chain variable region of SEQ ID NO: 53.

In another embodiment, the anti-4-1BB antibody or antigen-binding fragment thereof may be a scFv (single chain variable fragment) comprising:
a heavy chain variable region comprising a CDR-H1 comprising an amino acid sequence selected from SEQ ID NOs: 44 and 45, a CDR-H2 comprising an amino acid sequence selected from SEQ ID NOs: 46 and 47, and a CDR-H3 comprising an amino acid sequence selected from SEQ ID NOs: 48 to 51;
a light chain variable region comprising a CDR-L1 comprising an amino acid sequence of SEQ ID NO: 54, a CDR-L2 comprising an amino acid sequence of SEQ ID NO: 55, and a CDR-L3 comprising an amino acid sequence of SEQ ID NO: 56; and
a peptide liker between the heavy chain variable region and the light chain variable region.

More specifically, the anti-4-1BB antibody or antigen-binding fragment thereof may be a scFv comprising:
a heavy chain variable region comprising an amino acid sequence selected from the group consisting of SEQ ID NOs: 39 to 43;
a light chain variable region comprising an amino acid sequence selected from SEQ ID NOs: 52 and 53; and
a peptide liker between the heavy chain variable region and the light chain variable region.

For example, the anti-4-1BB antibody or fragment thereof may be a scFv comprising:
a heavy chain variable region comprising an amino acid sequence of selected from the group consisting of SEQ ID NOs: 39 to 43 or an amino acid sequence having a sequence identity of at least 80%, at least 85%, at least 90%, at least 95%, at least 96%, at least 97%, at least 98%, or at least 99% to the above described amino acid sequences; and
a light chain variable region comprising an amino acid sequence selected from SEQ ID NOs: 52 and 53, or an amino acid sequence having a sequence identity of at least 80%, at least 85%, at least 90%, at least 95%, at least 96%, at least 97%, at least 98%, or at least 99% to the above described amino acid sequences; and
a peptide liker between the heavy chain variable region and the light chain variable region.

For example, the anti-4-1BB antibody or antigen-binding fragment thereof may be a scFv comprising:
(1) a heavy chain variable region of SEQ ID NO: 39 and a light chain variable region of SEQ ID NO: 52;
(2) a heavy chain variable region of SEQ ID NO: 40 and a light chain variable region of SEQ ID NO: 52;
(3) a heavy chain variable region of SEQ ID NO: 41 and a light chain variable region of SEQ ID NO: 52;
(4) a heavy chain variable region of SEQ ID NO: 40 and a light chain variable region of SEQ ID NO: 53;
(5) a heavy chain variable region of SEQ ID NO: 41 and a light chain variable region of SEQ ID NO: 53;
(6) a heavy chain variable region of SEQ ID NO: 42 and a light chain variable region of SEQ ID NO: 52;
(7) a heavy chain variable region of SEQ ID NO: 43 and a light chain variable region of SEQ ID NO: 53; and
a peptide liker between the heavy chain variable region and the light chain variable region.

In the present disclosure, the anti-4-1BB scFv may comprise a heavy chain variable region and a light chain variable region, in any order. For example, the anti-4-1BB scFv may comprise a light chain variable region and a heavy chain variable region in order from N-terminus to C-terminus. Alternatively, the anti-4-1BB scFv may comprise a heavy chain variable region and a light chain variable region in order from N-terminus to C-terminus.

In an embodiment, in the scFv, the heavy chain variable region and the light chain variable region may be linked via a suitable peptide linker. For example, the peptide linker may be (GGGGS)2 which is SEQ ID NO: 72, (GGGGS)3 which is SEQ ID NO: 73, (GGGGS)4 which is SEQ ID NO: 68, or (GS)9 which is SEQ ID NO: 69.

Each of heavy chain variable regions and light chain variable regions disclosed herein may be combined with various constant regions of heavy chain and light chain to form heavy chain and light chain of an intact antibody, respectively.

These anti-4-1BB antibodies may be useful for therapeutic purposes such as treating various types of cancer, etc., and can also be used for diagnostic and prognostic purposes.

The antibodies of the disclosure are characterized by particular functional features or properties of the antibodies. For example, the antibodies specifically bind to human 4-1BB and may bind to 4-1BB originated from certain other species, e.g., monkey 4-1BB, e.g., cynomolgus monkey, rhesus monkey, but may not substantially bind to 4-1BB originated from certain other species, e.g., mouse 4-1BB. Preferably, an antibody of the disclosure binds to human 4-1BB with high affinity.

The binding of an antibody of the disclosure to 4-1BB can be assessed using one or more techniques well established in the art. For example, in a preferred embodiment, an antibody can be tested by a flow cytometry assay in which the antibody is reacted with a cell line that expresses human 4-1BB, such as CHO cells that have been transfected to express 4-1BB, e.g., human 4-1BB, or monkey 4-1BB, e.g., rhesus or cynomolgus monkey or mouse 4-1BB on their cell surface. Other suitable cells for use in flow cytometry assays include anti-CD3-stimulated CD4⁺ activated T cells, which express native 4-1BB. Still other suitable binding assays include ELISA assays, for example using a recombinant 4-1BB protein. Additionally, or alternatively, the binding of the antibody, including the binding kinetics (e.g., KD value) can be tested in Octet analysis. For example, binding affinities of an anti-4-1BB antibody or an antigen binding fragment thereof of the disclosure to human 4-1BB include those with a dissociation constant or KD of 1×10⁻⁶ M or less, 1×10⁻⁷ M or less, 1×10⁻⁸ M or less, 1×10⁻⁹ M or less, 1×10⁻¹⁰ M or less, or 1.80×10⁻¹⁰ M or less.

Without limitation, the anti-4-1BB antibody or fragment thereof, the anti-B7-H4 antibody or fragment thereof, and/or the bispecific antibody may be a chimeric antibody, a humanized antibody, or a fully human antibody. In one aspect, antibody or fragment thereof is not naturally occurring, or chemically or recombinantly synthesized. Given that each of these antibodies can bind to 4-1BB (such as, human 4-1BB) and/or to B7-H4 (such as, human B7-H4), the CDR sequences or the VH and VL sequences as described above can be “mixed and matched” to create other anti-4-1BB binding molecules and/or other anti-B7-H4 binding molecules. Preferably, when the CDRs sequences or VH and VL chains are mixed and matched, for example, a VH sequence from a particular VH/VL pairing is replaced with a structurally similar VH sequence. Likewise, preferably a VL sequence from a particular VH/VL pairing is replaced with a structurally similar VL sequence.

### Anti-B7-H4/Anti-4-1BB Bispecific Antibody

The bispecific antibody described herein may comprise:
(1) an anti-B7-H4 antibody or an antigen-binding fragment thereof, which is capable of specifically recognizing and/or binding to B7-H4 protein; and
(2) an anti-4-1BB antibody or an antigen-binding fragment thereof, which is capable of specifically recognizing and/or binding to 4-1BB protein.

The bispecific antibody can bind both of B7-H4 and 4-1BB. In the bispecific antibody, the anti-B7-H4 antibody or antigen-binding fragment thereof and the anti-4-1BB antibody or antigen-binding fragment thereof are as described above.

The bispecific antibody may possess advantages due to functions of an anti-B7-H4 antibody or its fragment and/or an anti-4-1BB antibody or its fragment. In certain embodiments, due to an anti-B7-H4 antibody or antigen-binding fragment thereof, the bispecific antibody may possess ability to enhance secretion of cytokines. In certain embodiments, due to an anti-4-1BB antibody or antigen-binding fragment thereof, the bispecific antibody may be capable of binding to human 4-1BB and exhibiting an ability to activate T cells. The anti-B7-H4/anti-4-1BB bispecific antibody may activate 4-1BB signaling under the condition of B7-H4-expressing tumor cells. In addition, the anti-4-1BB antibody or an antigen-binding fragment thereof contained in the bispecific antibody may be characterized by localizing and/or activating only in tumor microenvironment (TME), and/or considerably reducing liver toxicities compared to pre-existing anti-4-1BB antibodies, with maintaining the efficacies of immune response enhancement and/or tumor treatment.

Other means by which to evaluate the ability of the antibody to stimulate an immune response include the ability of the antibody to inhibit tumor growth, such as in an in vivo tumor graft model.

In the bispecific antibody comprising the B7-H4 targeting moiety and the 4-1BB targeting moiety, one of the B7-H4 targeting moiety and the 4-1BB targeting moiety can be a full-length antibody, and the other can be an antigen-binding fragment (e.g., scFv) comprising heavy chain CDRs, light chain CDRs, or a combination thereof, but it may not be limited thereto. The full-length antibody targeting one of B7-H4 and 4-1BB proteins, and the antigen-binding fragment targeting the other protein may be chemically linked (e.g., covalently linked) directly or via a peptide linker. The antigen-binding fragment (e.g., scFv) may be linked directly or via a peptide linker to N-terminus of the full-length antibody (e.g., N-terminus of a light chain or a heavy chain of the full-length antibody), C-terminus of the full-length antibody (e.g., C-terminus of a heavy chain (or Fc or CH3 domain) of the full-length antibody), or both thereof.

In an embodiment, the bispecific antibody may comprise a full-length anti-B7-H4 antibody, an antigen-binding fragment (e.g., scFv) of an anti-4-1BB antibody, and a peptide linker therebetween. In other embodiment, the bispecific antibody may comprise a full-length anti-4-1BB antibody, an antigen-binding fragment (e.g., scFv) of an anti-B7-H4 antibody, and a peptide linker therebetween.

In an embodiment, the scFv contained in the bispecific antibody may comprise a heavy chain variable region and a light chain variable region in any order. For example, the scFv contained in the bispecific antibody may comprise a heavy chain variable region and a light chain variable, in a direction from N-terminus to C-terminus, and optionally a peptide linker therebetween, or alternatively, the scFv contained in the bispecific antibody may comprise a light chain variable region and a heavy chain variable, in a direction from N-terminus to C-terminus, and optionally a peptide linker therebetween.

In an embodiment, the anti-B7-H4/anti-4-1BB bispecific antibody activates 4-1BB signaling, and as a result immune response, depending on B7-H4 expressed on cell surfaces.

When the bispecific antibody comprises a full-length anti-B7-H4 antibody and an anti-4-1BB scFv, the bispecific antibody may comprise:
(i) a first polypeptide comprising, in a direction from N-terminus to C-terminus:
a heavy chain of an anti-B7-H4 antibody,
optionally, a peptide linker (a first peptide linker), and
an anti-4-1BB scFv; and
(ii) a second polypeptide comprising a light chain of the anti-B7-H4 antibody,
wherein the anti-4-1BB scFv may comprise, in a direction from N-terminus to C-terminus:
a light chain variable region of an anti-4-1BB antibody,
optionally, a peptide linker (a second peptide linker), and
a heavy chain variable region of the anti-4-1BB antibody.

Alternatively, the bispecific antibody may comprise:
(i) a first polypeptide comprising, in a direction from N-terminus to C-terminus:
a heavy chain of an anti-B7-H4 antibody,
optionally, a peptide linker (a first peptide linker), and
an anti-4-1BB scFv; and
(ii) a second polypeptide comprising a light chain of the anti-B7-H4 antibody,
wherein the anti-4-1BB scFv may comprise, in a direction from N-terminus to C-terminus:
a heavy chain variable region of the anti-4-1BB antibody,
optionally, a peptide linker (a second peptide linker), and
a light chain variable region of an anti-4-1BB antibody.

Alternatively, the bispecific antibody may comprise:
(i) a first polypeptide comprising, in a direction from N-terminus to C-terminus:
an anti-4-1BB scFv,
optionally, a peptide linker (a first peptide linker), and
a heavy chain of an anti-B7-H4 antibody; and
(ii) a second polypeptide comprising a light chain of the anti-B7-H4 antibody,
wherein the anti-4-1BB scFv may comprise, in a direction from N-terminus to C-terminus:
a light chain variable region of an anti-4-1BB antibody,
optionally, a peptide linker (a second peptide linker), and
a heavy chain variable region of the anti-4-1BB antibody.

Alternatively, the bispecific antibody may comprise:
(i) a first polypeptide comprising, in a direction from N-terminus to C-terminus:
an anti-4-1BB scFv,
optionally, a peptide linker (a first peptide linker), and
a heavy chain of an anti-B7-H4 antibody; and
(ii) a second polypeptide comprising a light chain of the anti-B7-H4 antibody,
wherein the anti-4-1BB scFv may comprise, in a direction from N-terminus to C-terminus:
a heavy chain variable region of the anti-4-1BB antibody,
optionally, a peptide linker (a second peptide linker), and
a light chain variable region of an anti-4-1BB antibody.

In another embodiment, both of the B7-H4 targeting moiety and the 4-1BB targeting moiety contained in the bispecific antibody may be a full-length antibody or an antigen-binding fragment comprising heavy chain CDRs, light chain CDRs, or a combination thereof, which are linked to each other directly or via a peptide linker.

Given that each of antibodies can bind to both of 4-1BB (such as, human 4-1BB) and B7-H4 (such as, human B7-H4), the CDR sequences, or VH (heavy chain variable region) and VL (light chain variable region) sequences as disclosed herein can be “mixed and matched” to create other anti-B7-H4/anti-4-1BB binding bispecific molecules.

In another embodiment, both of the B7-H4 targeting moiety and the 4-1BB targeting moiety may be a full-length antibody or an antigen-binding fragment comprising heavy chain CDRs, light chain CDRs, or a combination thereof.

In another embodiment, the bispecific antibody may be in a heterodimeric form, which comprises a first arm comprising a pair of a first heavy chain and a first light chain targeting one of B7-H4 and 4-1BB, and a second arm comprising a pair of a second heavy chain and a second light chain targeting the other one.

In an embodiment, the full-length antibody may be in a full-length immunoglobulin form (e.g., IgG, IgM, IgA, IgE or IgD, such as, human IgG, human IgM, human IgA, human IgE, or human IgD), and the antigen-binding fragment may be selected from the group consisting of Fab, Fab′, F(ab′)2, Fd, Fv, scFv, single-chain antibodies, sdFv, and the like, as described above. For example, the full-length antibody may be in a full-length human IgG (human IgG1, human IgG2, human IgG3, or human IgG4) form, and the antigen-binding fragment may be scFv.

### Peptide Linker

The use of a peptide linker for the bispecific antibody may lead to a high purity of the antibody. That is, for high purity of the antibody, the bispecific antibody may comprise a peptide linker between a heavy chain and scFv in a first polypeptide (a first peptide linker), and/or between heavy and light variable regions in scFv (a second peptide linker).

As used herein, the term “peptide linker” may be those including any amino acids of 1 to 100, particularly 2 to 50, and any kinds of amino acids may be included without any restrictions. The peptide linker may include for example, Gly, Asn and/or Ser residues, and also include neutral amino acids such as Thr and/or Ala. Amino acid sequences suitable for the peptide linker may be those known in the relevant art. Meanwhile, a length of the peptide linker may be variously determined within such a limit that the functions of the polypeptide and/or scFv will not be affected. For instance, the peptide linker may be formed by including a total of about 1 to about 100, about 2 to about 50, or about 5 to about 25 (e.g., 1, 2, 3, 4, 5, 6, 7, 8, 9, 10, 11, 12, 13, 14, 15, 16, 17, 18, 19, 20, 21, 22, 23, 24, or 25) of one or more selected from the group consisting of Gly, Asn, Ser, Thr, and Ala. In one embodiment, the peptide linker may be represented as (GmSl)n (m, l, and n, are independently an integer of about 1 to about 10, particularly, 1, 2, 3, 4, 5, 6, 7, 8, 9, or 10). In one embodiment, the peptide linker can be amino acids of (GGGGS)2 which is SEQ ID NO: 72, (GGGGS)3 which is SEQ ID NO: 73, (GGGGS)4 which is SEQ ID NO: 68, or (GS)9 which is SEQ ID NO: 69, but not be limited thereto.

### Variable Antibodies

For example, an antibody described herein may comprise a flexible linker sequence, or may be modified to add a functional moiety (e.g., PEG, a drug, a toxin, or a label).

Antibodies or variants described herein may comprise derivatives that are modified, e.g., by the covalent attachment of any type of molecule to the antibody such that covalent attachment does not prevent the antibody from binding to the antigen (e.g., an epitope). For example, but not by way of limitation, the antibodies can be modified, e.g., by at least one selected from the group consisting of glycosylation, acetylation, pegylation, phosphorylation, phosphorylation, amidation, derivatization by known protecting/blocking groups, proteolytic cleavage, linkage to a cellular ligand or other protein, and the like. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

For some uses, including in vivo use of antibodies in humans and in vitro detection assays, it may be preferable to use chimeric, humanized, or human antibodies. Methods for producing chimeric antibodies or humanized antibodies are known in the art.

In another embodiment, DNA encoding desired antibodies may be readily isolated and sequenced using conventional procedures. The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins.

The present disclosure provides an isolated polynucleotide encoding the anti-B7-H4/anti-4-1BB bispecific antibody, the anti-B7-H4 antibody, the anti-4-1BB antibody or the antigen-binding fragment thereof.

In an embodiment an expression vector including the isolated polynucleotide may be provided.

In an embodiment, a hybridoma cell comprising the isolated polynucleotide encoding the antibody or the antigen-binding fragment of the present disclosure or the expression vector including the same may be provided.

Additionally, using routine recombinant DNA techniques, one or more of the CDRs of the antibody may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278: 457-479 (1998) for a listing of human framework regions). For example, the polynucleotide generated by the combination of the framework regions and CDRs encodes an antibody that specifically binds to at least one epitope of a desired polypeptide, e.g., LIGHT. Preferably, one or more amino acid substitutions may be made within the framework regions, and, preferably, the amino acid substitutions improve binding of the antibody to its antigen (or epitope). Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present disclosure and within the skill of the art.

Additionally, standard techniques known to those of skilled in the art can be used to introduce mutations in the nucleotide sequence encoding an antibody of the present disclosure.

As used herein, “Heavy Component” of an anti-B7-H4/anti-4-1BB bispecific antibody of the present disclosure may comprise (1) a heavy chain of anti-B7-H4 antibody and (2) a heavy chain variable region and light chain variable region of anti-4-1BB antibody when the bispecific antibody comprises an anti-B7-H4 full-length antibody and an anti-4-1BB scFv; or may comprise (1) a heavy chain of anti-4-1BB antibody and (2) a heavy chain variable region and light chain variable region of anti-B7-H4 antibody when the bispecific antibody comprises an anti-4-1BB full-length antibody and an anti-B7-H4 scFv.

As used herein, “Light Component” of an anti-B7-H4/anti-4-1BB bispecific antibody of the present disclosure may comprise: a light chain of anti-B7-H4 antibody when the bispecific antibody comprises an anti-B7-H4 full-length antibody and an anti-4-1BB scFv; or may comprise a light chain of anti-4-1BB antibody when the bispecific antibody comprises an anti-4-1BB full-length antibody and an anti-B7-H4 scFv.

### Therapeutic Use of the Bispecific Antibody

The bispecific antibody provided herein is capable of simultaneously bind to B7-H4 protein and the 4-1BB protein on the surface of cells, thereby exhibiting improved effects in immunotherapies and/or cancer therapies, for example, by activating immune response at the tumor microenvironment. Given the ability of the bispecific antibodies of the disclosure to bind to B7-H4 protein and to stimulate antigen-specific T cell responses, the disclosure also provides a composition or in vitro and in vivo methods of using the antibodies of the disclosure to stimulate, enhance or upregulate antigen-specific T cell responses.

An embodiment provides a pharmaceutical composition comprising the bispecific antibody or the anti-B7-H4 or anti-4-1BB antibody as described above. The pharmaceutical composition may further comprise a pharmaceutically acceptable carrier. The pharmaceutical composition may be used for stimulating an immune response (e.g., an antigen-specific T cell response), and/or treating and/or preventing a disease associated with B7-H4, 4-1BB, or both thereof.

Another embodiment provides the use of the bispecific antibody or the anti-B7-H4 antibody in the prevention or treatment of a disease related to B7-H4.

Another embodiment provides a method of stimulating an immune response (e.g., an antigen-specific T cell response), and/or treating and/or preventing a disease associated with B7-H4, 4-1BB, or both thereof, such as a cancer, in a subject in need thereof, comprising administering to the subject a pharmaceutically effective amount of the bispecific antibody or the pharmaceutical composition. The subject may be one in need of stimulating an immune response (e.g., an antigen-specific T cell response), and/or treating and/or preventing a disease associated with B7-H4, 4-1BB, or both thereof, such as a cancer. The method may further step of identifying the subject in need of stimulating an immune response, or treating and/or preventing a disease associated with B7-H4, 4-1BB, or both thereof, prior to the administering step.

Another embodiment provides a use of the bispecific antibody or the pharmaceutical composition in stimulating an immune response (e.g., an antigen-specific T cell response), and/or treating and/or preventing a disease associated with B7-H4, 4-1BB, or both thereof, such as a cancer. Another embodiment provides a use of the bispecific antibody in preparing a medicament for stimulating an immune response (e.g., an antigen-specific T cell response), and/or treating and/or preventing a disease associated with B7-H4, 4-1BB, or both thereof, such as a cancer.

In an embodiment, the subject may be selected from mammals including humans, monkeys, rats, mice, dogs, cats, guinea pigs, rabbits, rats, mice, horses, cattle, cows, and so on, or a cell or tissue obtained therefrom, but are not limited thereto. For example, the subject may be one in need of stimulating an immune response (e.g., an antigen-specific T cell response), and/or treating and/or preventing a disease associated with B7-H4, 4-1BB, or both thereof, such as a cancer. For example, the subject may be a mammal (e.g., a human) suffering from a cancer. In other embodiment, the subject may be a cell separated (isolated) from a mammal, for example, a mammal suffering from the disease selected from cancers infectious diseases, autoimmune reactions, nervous system disorders, and the like (e.g., a cancer cell or a cell separated (isolated) from an infectious region in the mammal, or a T cell, such as a tumor-infiltrating T lymphocyte, a CD4+ T cell, a CD8+ T cell, or the combination thereof). In a specific embodiment, the disease may be one associated with expression or high-expression (overexpression) of B7-H4, for example, the disease may be a cancer associated with expression or high-expression (overexpression) of B7-H4. For example, the “a cancer associated with high expression of B7-H4” may refer to a cancer related to a cancer cell which expresses B7-H4 higher than B7-H4 non-expressing cancer cell (such as cancer cell line SK-BR3, CAMA-1, etc.).

In the pharmaceutical compositions, methods and/or uses provided herein, the disease associated with B7-H4, 4-1BB, or both thereof may be one associated with activation (e.g., abnormal activation or over-activation) and/or overproduction (overexpression) of B7-H4, 4-1BB, or both thereof. For example, the disease may be a cancer or an infection.

The cancer may be a solid cancer or blood cancer. The cancer may be, but not limited to, one or more selected from the group consisting of breast cancer, colon cancer, gastric cancer, lung cancer (e.g., squamous cell carcinoma of the lung, small-cell lung cancer, non-small-cell lung cancer, adenocarcinoma of the lung), peritoneal carcinoma, skin cancer, squamous cell carcinoma, melanoma in the skin or eyeball, rectal cancer, cancer near the anus, esophagus cancer, small intestinal tumor, endocrine gland cancer, parathyroid cancer, adrenal cancer, soft-tissue sarcoma, urethral cancer, chronic or acute leukemia, lymphocytic lymphoma, hepatoma, gastrointestinal cancer, pancreatic cancer, glioblastoma, cervical cancer, ovarian cancer, liver cancer, bladder cancer, hepatocellular adenoma, large intestine cancer, endometrial carcinoma or uterine carcinoma, salivary gland tumor, kidney cancer, cervix cancer, prostate cancer, vulvar cancer, thyroid cancer, head and neck cancer, brain cancer, biliary tract cancer, gallbladder cancer, and the like. The cancer may be a primary cancer or a metastatic cancer.

As used herein, the term “prevention and/or treatment of cancer” may refer to cancer cell death, inhibition of cancer cell proliferation, alleviation of symptoms associated with cancer, inhibition of metastasis of cancer, etc.

As used herein, the term “stimulation of immune response” may refer to 4-1BB signal activation, enhancement in any immune response associated with 4-1BB, such as 4-1BB-induced signal activation (e.g., 4-1BB-induced NF-kB signal activation, increase in release of cytokine, target cell killing by immune cells, such as T cells, and the like, but not be limited thereto). In some embodiment, the enhancement of immune response by the bispecific antibody provided by this disclosure may occur be in the presence of B7-H4 (under the condition of B7-H4 expression).

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. The amount used can be determined by pharmacological and pharmacokinetic principles well known in the art.

The administration of the bispecific antibody or the anti-B7-H4 or anti-4-1BB antibody may be conducted through at least one selected from the group consisting of intraperitoneal, intravenous, subcutaneous, intradermal, intramuscular, intranasal, epidural, and oral routes, but not be limited thereto. The bispecific antibody or the anti-B7-H4 or anti-4-1BB antibody or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, parenterally, intracistemally, intravaginally, intraperitoneally, rectally, topically (as by powders, ointments, drops or transdermal patch), bucally, or as an oral or nasal spray. The term “parenteral” as used herein refers to modes of administration which include intravenous, intramuscular, intraperitoneal, intrasternal, Intratumoral, subcutaneous and intra-articular injection and infusion.

The pharmaceutically effective amount of the bispecific antibodies or the anti-B7-H4 or anti-4-1BB antibodies for treating, inhibiting, ameliorating, and/or preventing an inflammatory, immune or malignant disease, disorder, or condition, can be determined by standard clinical techniques.

The pharmaceutical compositions may comprise an effective amount of the bispecific antibody or the anti-B7-H4 or anti-4-1BB antibody, and an acceptable carrier. In some embodiments, the composition further includes a second anticancer agent (e.g., an immune checkpoint inhibitor).

### Diagnostic Use of the Antibody

Over-expression and/or over-activation of B7-H4 and/or 4-1BB is observed in a biological sample (e.g., cells, tissues, blood, serum, etc.) from a patient suffering from a certain cancer and/or infection (for example, tumor cell or tissue, blood or serum from an infectious patient), and/or patients having B7-H4- and/or 4-1BB-over-expressing cells are likely responsive to treatments with the bispecific antibody or the anti-B7-H4 or anti-4-1BB antibody. Accordingly, the bispecific antibody or the anti-B7-H4 or anti-4-1BB antibody of the present disclosure can also be used for diagnostic and prognostic purposes.

An embodiment provides a pharmaceutical composition for diagnosing a disease associated with B7-H4, 4-1BB, or both thereof, the composition comprising the bispecific antibody or the anti-B7-H4 or anti-4-1BB antibody. In another embodiment, provided is a use of the bispecific antibody or the anti-B7-H4 or anti-4-1BB antibody for diagnosing a disease associated with B7-H4, 4-1BB, or both thereof.

In the diagnosing method and/or detecting method, the step of detecting the antigen-antibody reaction or measuring a level of the antigen-antibody reaction may be performed by any general method known to the relevant art.

Another embodiment provides a method for providing diagnostic information regarding a disease related to B7-H4.

### Polynucleotides Encoding the Antibodies and Methods of Preparing the Antibodies

An embodiment provides a polynucleotide encoding the bispecific antibody or the anti-B7-H4 or anti-4-1BB antibody. In particular, an embodiment provides a polynucleotide encoding a heavy chain of the bispecific antibody in an IgG-scFv form. Other embodiment provides a polynucleotide encoding a light chain of the bispecific antibody in the IgG-scFv form. The IgG-scFv form may refer to a kind of a bispecific antibody comprising a full-length IgG antibody targeting (binding to) one of B7-H4 and 4-1BB proteins and a scFv fragment targeting (binding to) the other one, wherein the scFv is linked to a C-terminus and/or N-terminus of the full-length IgG antibody directly (without a peptide linker) or via a peptide linker.

In an embodiment, when the bispecific antibody in an IgG-scFv form comprises a full-length IgG antibody against B7-H4 and a scFv fragment against 4-1BB, the polynucleotide encoding a heavy chain of the bispecific antibody may encode a heavy chain of the full-length IgG antibody against B7-H4 and a scFv fragment against 4-1BB that is linked to a C-terminus and/or N-terminus of the full-length IgG antibody directly or via a peptide linker; and the polynucleotide encoding a light chain of the bispecific antibody may encode a light chain of the full-length IgG antibody against B7-H4.

In another embodiment, when the bispecific antibody in an IgG-scFv form comprises a full-length IgG antibody against 4-1BB and a scFv fragment against B7-H4, the polynucleotide encoding a heavy chain of the bispecific antibody may encode a heavy chain of the full-length IgG antibody against 4-1BB and a scFv fragment against B7-H4 that is linked to a C-terminus and/or N-terminus of the full-length IgG antibody directly or via a peptide linker; and the polynucleotide encoding a light chain of the bispecific antibody may encode a light chain of the full-length IgG antibody against 4-1BB.

Another embodiment provides a recombinant vector comprising the polynucleotide encoding a heavy chain of the bispecific antibody, the polynucleotide encoding a light chain of the bispecific antibody, or both thereof. Another embodiment provides a recombinant cell transfected with the recombinant vector.

In an embodiment, an expression vector comprising the isolated polynucleotide encoding the antibody or the antigen-binding fragment of the present disclosure.

Another embodiment provides a method of preparing the bispecific antibody, comprising expressing the polynucleotide encoding a heavy chain of the bispecific antibody, the polynucleotide encoding a light chain of the bispecific antibody in a cell. The step of expressing the polynucleotide may be conducted by culturing the cell comprising the polynucleotide (for example, in a recombinant vector) under a condition allowing the expression of the polynucleotide. The method may further comprise isolating and/or purifying the bispecific antibody from the cell culture, after the step of expressing or culturing.

### Advantageous Effects

The present disclosure relates to bispecific antibodies, each of which comprises an antibody specific to B7-H4 and an antibody specific to 4-1BB, and uses thereof. The anti-B7-H4/4-1BB bispecific antibody may possess high affinities to B7-H4 and/or 4-1BB, and be capable of enhancing immune response and/or treating tumor (cancer) in a mammal, with reduced liver toxicities. The present disclosure also relates to anti-B7-H4 monospecific antibody having a function of inhibiting checkpoint activity of B7-H4 by specifically binding and detecting B7-H4 expressed in cancer cells with high affinity.

### DESCRIPTION OF DRAWINGS

FIG. 1 is the result of antigen binding abilities of mouse anti-B7-H4 antibodies by FACS analysis.

FIG. 2 is the result of antigen binding ability of humanized antibody 16E3H3 M1 as measured by ELISA.

FIG. 3 is the result of cancer cell binding property of anti-B7-H4 antibody by FACS analysis.

FIG. 4 is the result of T cell checkpoint ligand activity of anti-B7-H4 antibody by measurement of IFN-γ secretion from T cells.

FIG. 5 is the result of T cell checkpoint ligand activity of anti-B7-H4 antibody with dose dependent manner by measurement of IFN-γ secretion from T cells.

FIG. 6 is the result of antigen binding ability of anti-B7-H4/anti-4-1BB bispecific antibody to human B7-H4 and 4-1BB proteins as measured by DACE.

FIG. 7 is the result of antigen binding ability of anti-B7-H4/anti-4-1BB bispecific antibody to monkey B7-H4 and 4-1BB proteins as measured by DACE.

FIG. 8 is the result of cancer cell binding property of anti-B7-H4/anti-4-1BB bispecific antibody by FACS analysis.

FIG. 9 is the result of binding ability of anti-B7-H4/anti-4-1BB bispecific antibody against B7-family proteins as measured by ELISA.

FIG. 10 is the result of T cell checkpoint ligand activity of anti-B7-H4/anti-4-1BB bispecific antibody by measurement of IFN-γ secretion from T cells.

FIG. 11 is the result of T cell checkpoint ligand activity of anti-B7-H4/anti-4-1BB bispecific antibody with dose dependent manner by measurement of IFN-γ secretion from T cells.

FIG. 12 is the result of PBMC based target cell lysis test of anti-B7-H4/anti-4-1BB bispecific antibody.

FIGS. 13A and 13B are the results of PBMC based target cell lysis test of anti-B7-H4/anti-4-1BB bispecific antibody using various PBMC lots.

FIG. 14 is the result of in vitro 4-1BB activity test of anti-B7-H4/anti-4-1BB bispecific antibody.

FIGS. 15 and 16 are the results of in vivo efficacy test (anti-tumor activity) of anti-B7-H4/anti-4-1BB bispecific antibody with dose dependent manner.

FIG. 17 is the result of re-challenge test (anti-tumor activity) of anti-B7-H4/anti-4-1BB bispecific antibody with dose dependent manner.

### MODE FOR INVENTION

One or more embodiments of the present disclosure will now be described in detail with reference to the following examples. However, these examples are only for illustrative purposes and are not intended to limit the scope of the one or more embodiments of the present disclosure.

### Preparation Example 1. Generation of Anti-B7-H4 Antibody

### 1-1. Mouse Monoclonal Antibody Generation by Immunization

Monoclonal antibodies against human B7-H4 were generated using the following procedures. SJL and Balb/C mice (Charles River Laboratories, Hollister, CA) were hyperimmunized with either 293 cells overexpressing recombinant human B7-H4.

Specifically, mice were injected with 293 cells overexpressing human B7-H4 in PBS (5 million/dose via intraperitoneal) followed by a protein boost with recombinant human B7-H4 ECD (4 Dg/dose via intraperitoneal). Serum titers were evaluated by standard enzyme linked immunosorbant assay (ELISA) and FACS following 6-9 injections. Splenic B cells harvested from sera B7-H4 positive mice were fused with mouse myeloma cells (X63.Ag8.653; American Type Culture Collection, Manassas, VA, USA) by electrofusion (Hybrimune; Harvard Apparatus, Inc., Holliston, MA, USA). After 10-14 days, hybridoma supernatants were screened for antibody secretion by ELISA. All positive clones were then expanded and re-screened for binding to huB7-H4 by ELISA and FACS.

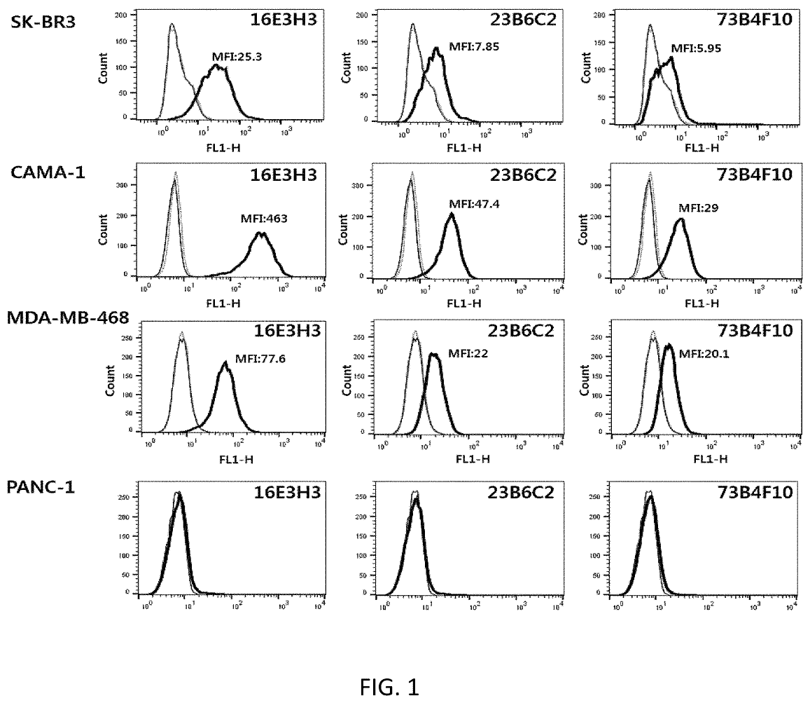

Three hybridoma clones were identified: 16E3H3, 23B6C2 and 73B4F10 (Identified from the B7-H4 immunized SJL mouse). All clones were positively bound to B7-H4 expressed cell line (CHOK-1 B7-H4, SK-BR-3) and recombinant human B7-H4 by fluorescent activated cell sorting (FACs) as shown in FIG. 1. Variable regions and CDRs of the identified mouse clones are as below:

TABLE 9

| 16E3H3 | | SEQ ID NO |
|---|---|---|
| Variable Heavy | QIQLVHSGPELKKPGETVKISCKASGYPFTTYGMNW | 22 |
| Chain | VKQAPGKGLKWMGWIDTYSGVPTYADDFKGRFAFS | |
| | LETSASTAYLQINNLKNEDTATYFCARRSFWYFDVWG | |
| | TGTTVTVSS | |
| HCDR1 | TYGMN | 2 |
| HCDR2 | WIDTYSGVPTYADDFKG | 3 |
| HCDR3 | RSFWYFDV | |
| Variable Light | DVVMTQTPLSLPVSLGDQASISCRSSQSLVHSNGNT | 23 |
| Chain | YLHWYLQKPGQSPKLLIYKVSNRFSGVPDRFSGSGS | |
| | GTDFTLKIRRVEAEDLGVYFCSQSTHVPWTFGGGTKL | |
| LCDR1 | RSSQSLVHSNGNTYCH | 7 |
| LCDR2 | KVSNRFS | 9 |
| LCDR3 | SQSTHVPWT | 10 |

TABLE 10

| 23B6C2 | | SEQ ID NO |
|---|---|---|
| Variable | QVTLKESGPGILQPSQTLSLTCSFSGFSLSTFGMGVGWIRQP | 24 |
| Heavy | SGKGLEWLAHIWWDDDKYYNSALKSRLTISKETSKNQIFLK | |
| Chain | IANVDTTDTATYYCVRKDYYGYRGFAYWGQGTLVTVSA | |
| HCDR1 | TFGMGVG | 25 |
| HCDR2 | HIWWDDDKYYNSALKS | 26 |
| HCDR3 | KDYYGYRGFAY | 27 |
| Variable | QLVLTQSSSASFSLGASAKLTCTLSSQHTTYTIEWYQQQPLK | 28 |
| Light | PPKYVMELKKDGSHSTGDGIPDRFSGSSSGADRYLCISNIQ | |
| Chain | PEDEAIYICGVGDTIKEQFVYVFGGGTKVTVL | |
| LCDR1 | TLSSQHTTYTIE | 29 |
| LCDR2 | LKKDGSHSTGD | 30 |
| LCDR3 | GVGDTIKEQFVYV | 31 |

TABLE 11

| 73B4F10 | | SEQ ID NO |
|---|---|---|
| Variable Heavy | QVQLQQPGTELVKPGASVKLSCKASGYTFTSYWMHW | 32 |
| Chain | VKQRPGQGLERIGHINPSNGGTNYNEKFKKKATLTVD | |
| | KSSSTAYMQLSSLTSEDSAVYYCARSEFYYGTVFAYWG | |
| | QGTLVTVSA | |
| HCDR1 | SYWMH | 33 |
| HCDR2 | HINPSNGGTNYNEKFKK | 34 |
| HCDR3 | SEFYYGTVFAY | 35 |
| Variable Light | DVLMTQTPLSLPVSLGDQASISCRSSQSIVHGNGNTYL | 36 |
| Chain | EWYLQKPGQSPKLLIYKVSNRFSGVPDRFSGSGSGTDF | |
| | TLKISRVEAEDLGVYYCFQGSHDPPTFGGGTKLEIK | |
| LCDR1 | RSSQSIVHGNGNTYLE | 37 |
| LCDR2 | KVSNRFS | 9 |
| LCDR3 | FQGSHDPPT | 38 |

### 1-2. Cloning and Chimerization of Mouse Monoclonal Antibodies

Mouse monoclonal antibodies 16E3H3, 23B6C2 and 73B4F10 were cloned and chimerized as follows.

Total RNA was extracted from hybridoma cells producing murine 16E3H3, 23B6C2 and 73B4F10 using standard methods. The variable light (VL) and variable heavy (VH) domains were amplified using RT-PCR with degenerate primers to the heavy and light chains. The forward primers were specific for the N-terminal amino acid sequence of the VL and VH regions. Respectively, the LC and HC reverse primers were designed to anneal to a region in the constant light (CL) and constant heavy domain 1 (CH1), which are highly conserved across species. The polynucleotide sequence of the inserts was determined using routine sequencing methods. Each antibody was chimerized by cloning the mouse heavy chain variable region onto a human IgG1 heavy chain constant region and cloning the light chain variable region onto a human kappa light chain constant region.

### 1-3. Humanization of Mouse Monoclonal Antibodies

The structure of parental antibody was modelled by computer-aided homology modelling program. Humanized antibodies were designed using CDR grafting plus back mutation. Briefly, the CDRs of parental antibodies were grafted into the human acceptors to obtain humanized light chains. Among three (3) chimerized clones, 16E3H3 was chosen as a lead clone. The sequences of humanized heavy and light chain of 16E3H3 clone are shown in Tables 12 and 13. The light chain was further modified to remove post-translational modification (PTM). The modified sequence of the light chain is shown in Table 14 (16E3H3 M1 clone). The constant region of the antibody contained in the antibody can still be modified by introducing more than one mutation or change (e.g., ADCC-reducing mutation (N297A mutation; Cancer Cell, vol. 19, issue 1, pp. 101-113, etc.)) into human IgG1.

TABLE 12
Example of Heavy Chain of B7-H4 IgG1 (16E3H3 and 16E3H3 M1)

| | Amino acid Sequence | SEQ ID NO |
|---|---|---|
| Full sequence | QVQLVQSGAEVKKPGASVKVSCKASGYPFTTYGMN | 11 |
| | WVKQAPGQGLEWMGWIDTYSGVPTYADDFKGRVT | |
| | MTRDTSISTAYMELSRLRSDDTAVYFCARRSFWYFDV | |
| | WGAGTTVTVSSÅSTKGPSVFPLAPSSKSTSGGTAALGC | |
| | LVKDYFPEPVTVSWNSGALTSGVHTFPAVLQSSGLYSL | |
| | SSVVTVPSSSLGTQTYICNVNHKPSNTKVDKKVEPKSC | |
| | DKTHTCPPCPAPELLGGPSVFLFPPKPKDTLMISRTPEV | |
| | TCVVVDVSHEDPEVKFNWYVDGVEVHNAKTKPREEQ | |
| | YASTYRVVSVLTVLHQDWLNGKEYKCKVSNKALPAPIE | |
| | KTISKAKGQPREPQVYTLPPSREEMTKNQVSLTCLVKG | |
| | FYPSDIAVEWESNGQPENNYKTTPPVLDSDGSFFLYSK | |
| | LTVDKSRWQQGNVFSCSVMHEALHNHYTQKSLSLSP | |
| | GK | |
| Heavy Chain | QVQLVQSGAEVKKPGASVKVSCKASGYPFTTYGMN | |
| Variable Region | WVKQAPGQGLEWMGWIDTYSGVPTYADDFKGRVT | |
| (VH) | MTRDTSISTAYMELSRLRSDDTAVYFCARRSFWYFDV | |
| | WGAGTTVTVSS | |
| Framework 1 | QVQLVQSGAEVKKPGASVKVSCKASGYPFT | 12 |
| CDR 1 | TYGMN | 2 |
| Framework 2 | WVKQAPGQGLEWMG | 13 |
| CDR 2 | WIDTYSGVPTYADDFKG | 3 |
| Framework 3 | RVTMTRDTSISTAYMELSRLRSDDTAVYFCAR | 14 |
| CDR 3 | RSFWYFDV | 4 |
| Framework 4 | WGAGTTVTVSS | 15 |

TABLE 13
Example of Light Chain of B7-H4 IgG1 (16E3H3)

| | Amino acid Sequence | SEQ ID NO |
|---|---|---|
| Full sequence | DVVMTQSPLSLPVTLGQPASISCRSSQSLVHSNGNT | 16 |
| | YLHWYLQRPGQSPRLLIYKVSNRFSGVPDRFSGSGS | |
| | GTDFTLKISRVEAEDVGVYFCSQSTHVPWTFGGGTK | |
| | VEIKRTVAAPSVFIFPPSDEQLKSGTASVVCLLNNFYP | |
| | REAKVQWKVDNALQSGNSQESVTEQDSKDSTYSLS | |
| | STLTLSKADYEKHKVYACEVTHQGLSSPVTKSFNRGE | |
| | C | |
| Light Chain | DWVMTQSPLSLPVTLGQPASISCRSSQSLVHSNGNT | 5 |
| Variable Region | YLHWYLQRPGQSPRLLIYKVSNRFSGVPDRFSGSGS | |
| (VL) | GTDFTLKISRVEAEDVGVYFCSQSTHVPWTFGGGTK | |
| | VEIKR | |
| Framework 1 | DVVMTQSPLSLPVTLGQPASISC | 17 |
| CDR 1 (WT) | RSSQSLVHSNGNTYCH | 7 |
| Framework 2 | WYLQRPGQSPRLLIY | 18 |
| CDR 2 | KVSNRFS | 9 |
| Framework 3 | GVPDRFSGSGSGTDFTLKISRVEAEDVGVYFC | 19 |
| CDR 3 | SQSTHVPWT | 10 |
| Framework 4 | FGGGTKVEIKR | 20 |

TABLE 14
Example of Light Chain of B7-H4 IgG1 (16E3H3 M1)

| | Amino acid Sequence | SEQ ID NO |
|---|---|---|
| Full sequence | DVVMTQSPLSLPVTLGQPASISCRSSQSLVHSNANT | 21 |
| | YLHWYLQRPGQSPRLLIYKVSNRFSGVPDRFSGSGS | |
| | GTDFTLKISRVEAEDVGVYFCSQSTHVPWTFGGGTK | |
| | VEIKRTVAAPSVFIFPPSDEQLKSGTASVVCLLNNFYP | |
| | REAKVQWKVDNALQSGNSQESVTEQDSKDSTYSLS | |
| | STLTLSKADYEKHKVYACEVTHQGLSSPVTKSFNRGE | |
| | C | |
| Light Chain | DVVMTQSPLSLPVTLGQPASISCRSSQSLVHSNANT | 6 |
| Variable Region | YLHWYLQRPGQSPRLLIYKVSNRFSGVPDRFSGSGS | |
| (VL) | GTDFTLKISRVEAEDVGVYFCSQSTHVPWTFGGGTK | |
| | VEIKR | |
| Framework 1 | DVVMTQSPLSLPVTLGQPASISC | 17 |
| CDR 1 (G29A | RSSQSLVHSNANTYLH | 8 |
| mutation for | | |
| PTM removal) | | |
| Framework 2 | WYLQRPGQSPRLLIY | 18 |
| CDR 2 | KVSNRFS | 9 |
| Framework 3 | GVPDRFSGSGSGTDFTLKISRVEAEDVGVYFC | 19 |
| CDR 3 | SQSTHVPWT | 10 |
| Framework 4 | FGGGTKVEIKR | 20 |

### Preparation Example 2. Generation of Anti-4-1BB Antibody

### 2-1. Preparation of Full Human Anti-4-1BB Monoclonal Antibodies

Full human anti-4-1BB monoclonal antibodies were prepared as disclosed in WO2020/111913. Specifically, for panning of a phage library (obtained from KBio Health) against target molecules, A total of four rounds of panning were carried out using 4-1BB (NCBI Accession No. NP_001552.2) coated immunotubes. Bacterial colonies from the 3 rounds of panning output were grown in SB-Carbenicilin in 96 deepwell plate until turbid, at which point 10¹¹ pfu of VCSM13 helper phage was added to each well. After 1 h infection at 37° C. with gentle shaking (80 rpm), 70 μg/mL of kanamycin was added, and the cells were cultured overnight at 30° C. with shaking at 200 rpm. Next day, the plates were centrifuged and the supernatants containing the phages were added to 4-1BB antigen-coated ELISA plates blocked with 3% BSA in PBST. After 1 h incubation at room temperature, the plates were washed three times with PBST and anti M13 antibody was added. The plates were incubated for 1 h, washed three times with PBST, and the binding activity was measured using tetramethylbenzidine (TMB).

The 4-1BB specific binders were amplified for plasmid DNA sequencing. Ig light chain V genes (VL) and VH sequences were analyzed to identify unique sequences and determine sequence diversity.

2-2. Preparation of Anti-4-1BB scFv Antibodies

Anti-4-1BB scFv antibodies with a structure of (N′)-VL-linker-VH-(C′) were prepared using the variable regions of the full human monoclonal antibodies against 4-1BB obtained in Preparation Example 2-1 above, wherein the amino acid residue “G” at the position 44 of a heavy chain variable region was substituted with “C”, and the amino acid residue “G” at the position 103 of a light chain variable region was substituted with “C”. Such amino acid substitution from “G” to “C” in scFv can contribute to increase in stabilities of bispecific antibodies comprising the scFv as one target-specific moiety. The amino acid sequences of the prepared anti-4-1BB scFvs were illustrated in Tables 15 to 21, while skilled persons in the art may apply changes or modifications of amino acid sequences to meet specific purposes, including applying various types of peptide linkers such as (GGGGS)2 which is SEQ ID NO: 72, (GGGGS)3 which is SEQ ID NO: 73, (GGGGS)4 which is SEQ ID NO: 68, or (GS)9 which is SEQ ID NO: 69.

TABLE 15
4-1BB scFv (1A10)

| | Amino acid Sequence | SEQ ID NO |
|---|---|---|
| Heavy Chain | EVQLLESGGGLVQPGGSLRLSCAASGFTFSSYDMS | 39 |
| Variable Region | WVRQAPGKCLEWVSWISYSGGSIYYADSVKGRFTI | |
| (VH) | SRDNSKNTLYLQMNSLRAEDTAVYYCARDGQRNS | |
| | MREFDYWGQGTLVTVSS | |
| Framework 1 | EVQLLESGGGLVQPGGSLRLSCAASGFTFS | 57 |
| CDR 1 | SYDMS | 44 |
| Framework 2 | WVRQAPGKCLEWVS | 58 |
| CDR 2 | WISYSGGSIYYADSVKG | 46 |
| Framework 3 | RFTISRDNSKNTLYLQMNSLRAEDTAVYYCAR | 59 |
| CDR 3 | DGQRNSMREFDY | 48 |
| Framework 4 | WGQGTLVTVSS | 60 |
| Linker between | GGGGSGGGGSGGGGSGGGGS | 68 |
| VL and VH | | |
| Light Chain | QSVLTOPPSASGTPGRRVTISCSGSSSNIGNNYVT | 52 |
| Variable Region | WYQQLPGTAPKLLIYADSHRPSGVPDRFSGSKSGT | |
| (VL) | SASLAISGLRSEDEADYYCATWDYSLSGYVFGCGT | |
| | KLTVL | |
| Framework 1 | QSVLTQPPSASGTPGRRVTISC | 61 |
| CDR 1 | SGSSSNIGNNYVT | 54 |
| Framework 2 | WYQQLPGTAPKLLIY | 62 |
| CDR 2 | ADSHRPS | 55 |
| Framework 3 | GVPDRFSGSKSGTSASLAISGLRSEDEADYYC | 63 |
| CDR 3 | ATWDYSLSGYV | 56 |
| Framework 4 | FGCGTKLTVL | 64 |

TABLE 16
4-1BB scFv (1A10M4)

| | Amino acid Sequence | SEQ ID NO |
|---|---|---|
| Heavy Chain | EVQLLESGGGLVQPGGSLRLSCAASGFTFSSYDMSWVR | 40 |
| Variable Region | QAPGKCLEWVSWISYSGGSIYYADSVKGRFTISRDNSKN | |
| (VH) | TLYLQMNSLRAEDTAVYYCARDAQRNSMREFDYWGQ | |
| | GTLVTVSS | |
| Framework 1 | EVQLLESGGGLVQPGGSLRLSCAASGFTFS | 57 |
| CDR 1 | SYDMS | 44 |
| Framework 2 | WVRQAPGKCLEWVS | 58 |
| CDR 2 | WISYSGGSIYYADSVKG | 46 |
| Framework 3 | RFTISRDNSKNTLYLQMNSLRAEDTAVYYCAR | 59 |
| CDR 3 | DAQRNSMREFDY | 49 |
| Framework 4 | WGQGTLVTVSS | 60 |
| Linker between | GGGGSGGGGSGGGGSGGGGS | 68 |
| VL and VH | | |
| Light Chain | QSVLTQPPSASGTPGRRVTISCSGSSSNIGNNYVTWYQ | 52 |
| Variable Region | QLPGTAPKLLIYADSHRPSGVPDRFSGSKSGTSASLAISG | |
| (VL) | LRSEDEADYYCATWDYSLSGYVFGCGTKLTVL | |
| Framework 1 | QSVLTQPPSASGTPGRRVTISC | 61 |
| CDR 1 | SGSSSNIGNNYVT | 54 |
| Framework 2 | WYQQLPGTAPKLLIY | 62 |
| CDR 2 | ADSHRPS | 55 |
| Framework 3 | GVPDRFSGSKSGTSASLAISGLRSEDEADYYC | 63 |
| CDR 3 | ATWDYSLSGYV | 56 |
| Framework 4 | FGCGTKLTVL | 64 |

TABLE 17
4-1BB scFv (1A10M11)

| | Amino acid Sequence | SEQ ID NO |
|---|---|---|
| Heavy Chain | EVQLLESGGGLVQPGGSLRLSCAASGFTFSSYDMSWV | 41 |
| Variable Region | RQAPGKCLEWVSWISYSGGSIYYADSVKGRFTISRDNS | |
| (VH) | KNTLYLOMNSLRAEDTAVYYCARDAQRQSMREFDYW | |
| | GQGTLVTVSS | |
| Framework 1 | EVQLLESGGGLVQPGGSLRLSCAASGFTFS | 57 |
| CDR 1 | SYDMS | 44 |
| Framework 2 | WVRQAPGKCLEWVS | 58 |
| CDR 2 | WISYSGGSIYYADSVKG | 46 |
| Framework 3 | RFTISRDNSKNTLYLQMNSLRAEDTAVYYCAR | 59 |
| CDR 3 | DAQRQSMREFDY | 50 |
| Framework 4 | WGQGTLVTVSS | 60 |
| Linker between | GGGGSGGGGSGGGGSGGGGS | 68 |
| VL and VH | | |
| Light Chain | QSVLTQPPSASGTPGRRVTISCSGSSSNIGNNYVTWYQ | 52 |
| Variable Region | QLPGTAPKLLIYADSHRPSGVPDRFSGSKSGTSASLAISG | |
| (VL) | LRSEDEADYYCATWDYSLSGYVFGCGTKLTVL | |
| Framework 1 | QSVLTQPPSASGTPGRRVTISC | 61 |
| CDR 1 | SGSSSNIGNNYVT | 54 |
| Framework 2 | WYQQLPGTAPKLLIY | 62 |
| CDR 2 | ADSHRPS | 55 |
| Framework 3 | GVPDRFSGSKSGTSASLAISGLRSEDEADYYC | 63 |
| CDR 3 | ATWDYSLSGYV | 56 |
| Framework 4 | FGCGTKLTVL | 64 |

TABLE 18
4-1BB scFv (1A10M12)

| | Amino acid Sequence | SEQ ID NO |
|---|---|---|
| Heavy Chain | EVQLLESGGGLVQPGGSLRLSCAASGFTFSSYDMS | 40 |
| Variable Region | WVRQAPGKCLEWVSWISYSGGSIYYADSVKGRFTI | |
| (VH) | SRDNSKNTLYLQMNSLRAEDTAVYYCARDAQRNS | |
| | MREFDYWGQGTLVTVSS | |
| Framework 1 | EVQLLESGGGLVQPGGSLRLSCAASGFTFS | 57 |
| CDR 1 | SYDMS | 44 |
| Framework 2 | WVRQAPGKCLEWVS | 58 |
| CDR 2 | WISYSGGSIYYADSVKG | 46 |
| Framework 3 | RFTISRDNSKNTLYLQMNSLRAEDTAVYYCAR | 59 |
| CDR 3 | DAQRNSMREFDY | 49 |
| Framework 4 | WGQGTLVTVSS | 60 |
| Linker between | GGGGSGGGGSGGGGSGGGGS | 68 |
| VL and VH | | |
| Light Chain | QSVLTQPPSASGTPGQRVTISCSGSSSNIGNNYVT | 53 |
| Variable Region | WYQQLPGTAPKLLIYADSHRPSGVPDRFSGSKSGT | |
| (VL) | SASLAISGLRSEDEADYYCATWDYSLSGYVFGCGT | |
| | KLTVL | |
| Framework 1 | QSVLTQPPSASGTPGQRVTISC | 65 |
| CDR 1 | SGSSSNIGNNYVT | 54 |
| Framework 2 | WYQQLPGTAPKLLIY | 62 |
| CDR 2 | ADSHRPS | 55 |
| Framework 3 | GVPDRFSGSKSGTSASLAISGLRSEDEADYYC | 63 |
| CDR 3 | ATWDYSLSGYV | 56 |
| Framework 4 | FGCGTKLTVL | 64 |

TABLE 19
4-1BB scFv (1A10M13)

| | Amino acid Sequence | SEQ ID NO |
|---|---|---|
| Heavy Chain | EVQLLESGGGLVQPGGSLRLSCAASGFTFSSYDMS | 41 |
| Variable Region | WVRQAPGKCLEWVSWISYSGGSIYYADSVKGRFTI | |
| (VH) | SRDNSKNTLYLQMNSLRAEDTAVYYCARDAQRQS | |
| | MREEDYWGQGTLVTVSS | |
| Framework 1 | EVQLLESGGGLVQPGGSLRLSCAASGFTFS | 57 |
| CDR 1 | SYDMS | 44 |
| Framework 2 | WVRQAPGKCLEWVS | 58 |
| CDR 2 | WISYSGGSIYYADSVKG | 46 |
| Framework 3 | RFTISRDNSKNTLYLQMNSLRAEDTAVYYCAR | 59 |
| CDR 3 | DAQRQSMREFDY | 50 |
| Framework 4 | WGQGTLVTVSS | 60 |
| Linker between | GGGGSGGGGSGGGGSGGGGS | 68 |
| VL and VH | | |
| Light Chain | QSVLTQPPSASGTPGQRVTISCSGSSSNIGNNYVT | 53 |
| Variable Region | WYQQLPGTAPKLLIYADSHRPSGVPDRFSGSKSGT | |
| (VL) | SASLAISGLRSEDEADYYATWDYSLSGYVFGCGT | |
| | KLTVL | |
| Framework 1 | QSVLTQPPSASGTPGQRVTISC | 65 |
| CDR 1 | SGSSSNIGNNYVT | 54 |
| Framework 2 | WYQQLPGTAPKLLIY | 62 |
| CDR 2 | ADSHRPS | 55 |
| Framework 3 | GVPDRFSGSKSGTSASLAISGLRSEDEADYYC | 63 |
| CDR 3 | ATWDYSLSGYV | 56 |
| Framework 4 | FGCGTKLTVL | 64 |

TABLE 20
4-1BB scFv (1A12)

| | Amino acid Sequence | SEQ ID NO |
|---|---|---|
| Heavy Chain | EVQLLESGGGLVQPGGSLRLSCAASGFTFSGYDMS | 42 |
| Variable Region | WVRQAPGKCLEWVSVIYPDDGNTYYADSVKGRFT | |
| (VH) | ISRDNSKNTLYLQMNSLRAEDAAVYYCAKHGGQK | |
| | PTTKSSSAYGMDGWGQGTLVTVSS | |
| Framework 1 | EVQLLESGGGLVQPGGSLRLSCAASGFTFS | 57 |
| CDR 1 | GYDMS | 45 |
| Framework 2 | WVRQAPGKCLEWVS | 58 |
| CDR 2 | VIYPDDGNTYYADSVKG | 47 |
| Framework 3 | RFTISRDNSKNTLYLQMNSLRAEDAAVYYCAK | 66 |
| CDR 3 | HGGQKPTTKSSSAYGMDG | 51 |
| Framework 4 | WGQGTLVTVSS | 60 |
| Linker between | GGGGSGGGGSGGGGSGGGGS | 68 |
| VL and VH | | |
| Light Chain | QSVLTQPPSASGTPGRRVTISCSGSSSNIGNNYVT | 52 |
| Variable Region | WYQQLPGTAPKLLIYADSHRPSGVPDRFSGSKSGT | |
| (VL) | SASLAISGLRSEDEADYYCATWDYSLSGYVFGCGT | |
| | KLTVL | |
| Framework 1 | QSVLTQPPSASGTPGRRVTISC | 61 |
| CDR 1 | SGSSSNIGNNYVT | 54 |
| Framework 2 | WYQQLPGTAPKLLIY | 62 |
| CDR 2 | ADSHRPS | 55 |
| Framework 3 | GVPDRFSGSKSGTSASLAISGLRSEDEADYYC | 63 |
| CDR 3 | ATWDYSLSGYV | 56 |
| Framework 4 | FGCGTKLTVL | 64 |

TABLE 21
4-1BB scFv (1A12M1)

| | Amino acid Sequence | SEQ ID NO |
|---|---|---|
| Heavy Chain | EVQLLESGGGLVQPGGSLRLSCAASGFTFSGYDMSW | 43 |
| Variable Region | VRQAPGKCLEWVSVIYPDDGNTYYADSVKGRFTISR | |
| (VH) | DNSKNTLYLQMNSLRAEDTAVYYCAKHGGQKPTTKS | |
| | SSAYGMDGWGQGTLVTVSS | |
| Framework 1 | EVQLLESGGGLVQPGGSLRLSCAASGFTFS | 57 |
| CDR 1 | GYDMS | 45 |
| Framework 2 | WVRQAPGKCLEWVS | 58 |
| CDR 2 | VIYPDDGNTYYADSVKG | 47 |
| Framework 3 | RFTISRDNSKNTLYLOMNSLRAEDTAVYYCAK | 67 |
| CDR 3 | HGGQKPTTKSSSAYGMDG | 51 |
| Framework 4 | WGQGTLVTVSS | 60 |
| Linker between | GGGGSGGGGSGGGGSGGGGS | 68 |
| VL and VH | | |
| Light Chain | QSVLTQPPSASGTPGQRVTISCSGSSSNIGNNYVTW | 53 |
| Variable Region | YQQLPGTAPKLLIYADSHRPSGVPDRFSGSKSGTSASL | |
| (VL) | AISGLRSEDEADYCATWDYSLSGYVFGCGTKLTVL | |
| Framework 1 | QSVLTQPPSASGTPGQRVTISC | 65 |
| CDR 1 | SGSSSNIGNNYVT | 54 |
| Framework 2 | WYQQLPGTAPKLLIY | 62 |
| CDR 2 | ADSHRPS | 55 |
| Framework 3 | GVPDRFSGSKSGTSASLAISGLRSEDEADYYC | 63 |
| CDR 3 | ATWDYSLSGYV | 56 |
| Framework 4 | FGCGTKLTVL | 64 |

TABLE 22
Linker between IgG1 and scFv

| Linker | Amino acid Sequence | SEQ ID NO |
|---|---|---|
| (GGGGS)4 | GGGGSGGGGSGGGGSGGGGS | 68 |
| (GS)9 | GSGSGSGSGSGSGSGSGS | 69 |

2-3. Preparation of Anti-4-1BB scFv Antibodies in the Form of Bispecific Antibody

As a representative example of a bispecific antibody, anti-B7-H4×4-1BB bispecific antibodies consisting of heavy components and light components as follows, were prepared:
(1) Heavy components (N′→C′)
1) heavy chain of anti-B7-H4 antibody;
2) linker: (GS)9; and
3) anti-4-1BB scFv prepared in Preparation Example 2-2
(2) Light components (N′→C′)
Light chain of anti-B7-H4 antibody

### Preparation Example 3. Generation of Anti-B7-H4/Anti-4-1BB Bispecific Antibody

The anti-B7-H4 IgG and anti-4-1BB scFv clones prepared in Preparation Example 1 and Preparation Example 2, respectively, were exemplarily selected, to prepare anti-B7-H4/anti-4-1BB bispecific antibodies in a IgG-scFv fusion form, in which a scFv antibody fragment of one antigen being fused to the C-terminal of IgG of another antigen. When B7-H4 is placed in full IgG part, IgG1 with ADCC reduced mutant backbone (N297A mutation) was used, and when 4-1BB is placed in full IgG part, IgG4 was used.

Especially, anti-B7-H4/anti-4-1BB bispecific antibody candidates have been prepared in full-length IgG (anti-B7-H4 antibody)-scFv(anti-4-1BB antibody) format. The constant region of the anti-B7-H4 antibody contained in the bispecific antibody was modified by introducing NA mutation (N297A). The amino acid sequences of the bispecific antibodies (16E3H3×1A10 M12 and 16E3H3 M1×1A10 M12) prepared according to this preparation example are presented in Table 23 and Table 24, respectively.

Hereinafter, 16E3H3 M1 monospecific antibody is also referred to as M40413, and 16E3H3 M1×1A10 M12 bispecific antibody is also referred to as B10317.

TABLE 23

| | 16E3H3 x1A10 M12 | SEQ ID NO |
|---|---|---|
| Heavy chain | QVQLVQSGAEVKKPGASVKVSCKASGYPFTTYGMN | 70 |
| | WVKQAPGQGLEWMGWIDTYSGVPTYADDFKGRVT | |
| | MTRDTSISTAYMELSRLRSDDTAVYFCARRSFWYFDV | |
| | WGAGTTVTVSSASTKGPSVFPLAPSSKSTSGGTAALGC | |
| | LVKDYFPEPVTVSWNSGALTSGVHTFPAVLQSSGLYSL | |
| | SSVVTVPSSSLGTQTYICNVNHKPSNTKVDKKVEPKSC | |
| | DKTHTCPPCPAPELLGGPSVFLFPPKPKDTLMISRTPEV | |
| | TCVVVDVSHEDPEVKFNWYVDGVEVHNAKTKPREEQ | |
| | YASTYRVVSVLTVLHQDWLNGKEYKCKVSNKALPAPIE | |
| | KTISKAKGQPREPQVYTLPPSREEMTKNQVSLTCLVKG | |
| | FYPSDIAVEWESNGQPENNYKTTPPVLDSDGSFFLYSK | |
| | LTVDKSRWQQGNVFSCSVMHEALHNHYTQKSLSLSP | |
| | GKGSGSGSGSGSGSGSGSGSQSVLTQPPSASGTPGQ | |
| | RVTISCSGSSSNIGNNYVTWYQQLPGTAPKLLIYADSH | |
| | RPSGVPDRFSGSKSGTSASLAISGLRSEDEADYYCATW | |
| | DYSLSGYVFGCGTKLTVLGGGGSGGGGSGGGGSGGG | |
| | GSEVQLLESGGGLVQPGGSLRLSCAASGFTFSSYDMS | |
| | WVRQAPGKCLEWVSWISYSGGSIYYADSVKGRFTISR | |
| | DNSKNTLYLOMNSLRAEDTAVYYCARDAQRNSMREF | |
| | DYWGQGTLVTVSS | |
| Light chain | DVVMTQSPLSLPVTLGQPASISCRSSQSLVHSNGNTYL | 16 |
| | HWYLQRPGQSPRLLIYKVSNRFSGVPDRFSGSGSGTD | |
| | FTLKISRVEAEDVGVYFCSQSTHVPWTFGGGTKVEIKR | |
| | TVAAPSVFIFPPSDEQLKSGTASVVCLLNNFYPREAKV | |
| | QWKVDNALQSGNSQESVTEQDSKDSTYSLSSTLTLSK | |
| | ADYEKHKVYACEVTHQGLSSPVTKSFNRGEC | |

TABLE 24

| | 16E3H3 M1 x1A10 M12 (B10317) | SEQ ID NO |
|---|---|---|
| Heavy chain | QVQLVQSGAEVKKPGASVKVSCKASGYPFTTYGM | 71 |
| | NWVKQAPGQGLEWMGWIDTYSGVPTYADDFKG | |
| | RVTMTRDTSISTAYMELSRLRSDDTAVYFCARRSFW | |
| | YFDVWGAGTTVTVSSASTKGPSVFPLAPSSKSTSG | |
| | GTAALGCLVKDYFPEPVTVSWNSGALTSGVHTFPA | |
| | VLQSSGLYSLSSVVTVPSSSLGTQTYICNVNHKPSN | |
| | TKVDKKVEPKSCDKTHTCPPCPAPELLGGPSVFLFP | |
| | PKPKDTLMISRTPEVTCVVVDVSHEDPEVKFNWYV | |
| | DGVEVHNAKTKPREEQYASTYRVVSVLTVLHQDW | |
| | LNGKEYKCKVSNKALPAPIEKTISKAKGQPREPQVY | |
| | TLPPSREEMTKNQVSLTCLVKGFYPSDIAVEWESNG | |
| | QPENNYKTTPPVLDSDGSFFLYSKLTVDKSRWQQG | |
| | NVFSCSVMHEALHNHYTQKSLSLSPGKGSGSGSG | |
| | SGSGSGSGSGSQSVLTQPPSASGTPGQRVTISCSG | |
| | SSSNIGNNYVTWYQQLPGTAPKLLIYADSHRPSGV | |
| | PDRFSGSKSGTSASLAISGLRSEDEADYYCATWDYS | |
| | LSGYVFGCGTKLTVLGGGGSGGGGSGGGGSGGGG | |
| | SEVQLLESGGGLVQPGGSLRLSCAASGFTFSSYDM | |
| | SWVRQAPGKCLEWVSWISYSGGSIYYADSVKGRFT | |
| | ISRDNSKNTLYLQMNSLRAEDTAVYYCARDAQRNS | |
| | MREFDYWGQGTLVTVSS | |
| Light chain | DVVMTQSPLSLPVTLGQPASISCRSSQSLVHSNAN | 21 |
| | TYLHWYLQRPGQSPRLLIYKVSNRFSGVPDRFSGS | |
| | GSGTDFTLKISRVEAEDVGVYFCSQSTHVPWTFGG | |
| | GTKVEIKRTVAAPSVFIFPPSDEQLKSGTASVVCLLN | |
| | NFYPREAKVQWKVDNALQSGNSQESVTEQDSKD | |
| | STYSLSSTLTLSKADYEKHKVYACEVTHQGLSSPVTK | |
| | SFNRGEC | |

Cloning of the bispecific antibody was performed as follows: A DNA segment 1 having a nucleotide sequence encoding a heavy chain of an IgG antibody of the anti-B7-H4/anti-4-1BB bispecific antibody was inserted into pcDNA 3.4 (Invitrogen, A14697; plasmid 1), and a DNA segment 2 having a nucleotide sequence encoding a light chain of an IgG antibody of the anti-B7-H4/anti-4-1BB bispecific antibody was inserted into pcDNA 3.4 (Invitrogen, A14697; plasmid 2). Thereafter, a DNA segment 3 encoding a scFv was fused at a part of the DNA segment 1 corresponding to the c-terminus of the Fc region of the IgG antibody inserted into the plasmid 1, using a DNA segment 4 encoding a linker peptide having 16 amino acid lengths consisting of (GGGGS)4 (SEQ ID NO: 68) or using a DNA segment 5 encoding a linker peptide having 18 amino acid lengths consisting of (GS)9 (SEQ ID NO: 69), to construct vectors for the expression of bispecific antibodies. Furthermore, in order to stabilize scFv, as described in Preparation Example 2, additional modification was applied to generate disulfide bridge fusing VL103-VH44(VL103: VL having G→C mutation at the position 103; VH 44: VH having G→C mutation at the position 44) to C-terminus of light chain and C-terminus of heavy chain, respectively.

### Experimental Example 1. Antigen Binding Abilities of Anti-B7-H4 Antibodies

### 1-1. Binding Confirmation of Mouse Anti-B7-H4 Antibody by Using FACS Analysis

To evaluate the antigen binding property, the mouse antibody candidates generated according to Preparation Example 1-1 were analyzed for their bindings to B7-H4 expressed cells (SK-BR3, CAMA-1 and MDA-MB-468) or B7-H4 negative expressed cell (PANC-1). Briefly, each cells were treated with the 100 nM of indicated antibodies at 4° C. for 1 hr. After washing by FACS buffer (1% BSA in PBS), Cells were incubated with the FITC anti-mouse IgG Fc antibody at 4° C. for 1 hr and then subjected to FACS analysis. As shown FIG. 1, anti-B7-H4 mouse antibodies bound to B7-H4 expressed SK-BR3, CAMA-1 and MDA-MB-468 cell lines but not B7-H4 negative PANC-1. In the results, among three (3) mouse antibody candidates, 16E3H3 clone showed highest binding affinity to B7-H4 expressed cancer cells. Based on the ranking results, 16E3H3 clone was selected for humanization.

### 1-2. Antigen Binding Ability of Anti-B7-H4 Monospecific Antibody to Human B7-H4 Protein

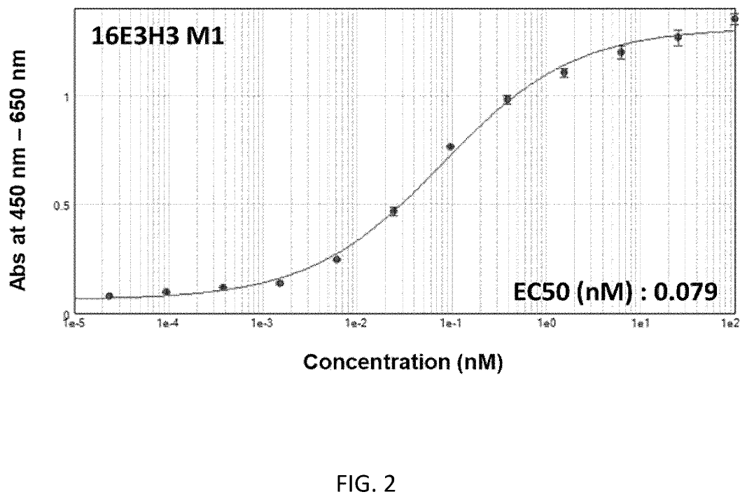

To evaluate the antigen binding activity of the humanized antibody, 16E3H3 M1 was subjected to ELISA. Briefly, ELISA plates were coated with Fc fused human B7-H4 protein at 1 μg/ml in PBS, 100 ul/well at 4° C. overnight, then blocked with 200 ul/well of 1% BSA in PBS at 37° C. Four fold dilutions of monoclonal antibody starting from 100 nM were added to each well and incubated for 1 hours at 37° C. The plates were washed with PBST (0.05% Tween 20 in PBS) and then incubated with HRP (Horse Radish Peroxidase) conjugated Anti-human Fab for 1 hr at 37° C. The plates were washed with PBST (Tween 20 0.05% in PBS) developed with TMB substrate and analyzed by spectrophotometer at OD 450 nm˜650 nm. As shown FIG. 2. 16E3H3 M1 bound to human B7-H4 with dose dependent manner. EC50 value was 0.079 nM.

### Experimental Example 2. Cell Binding Ability of Anti-B7-H4 Monospecific Antibody

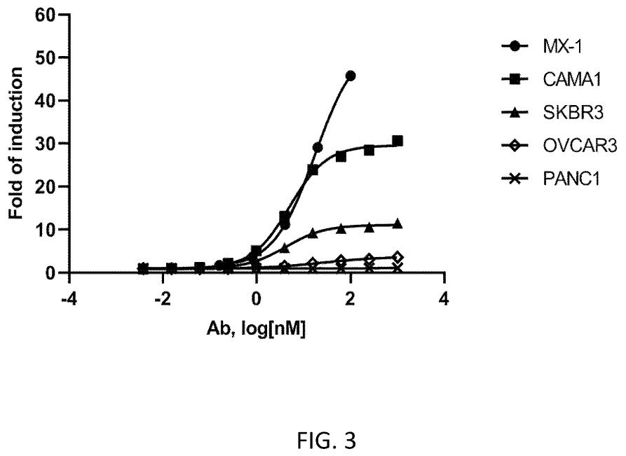

To evaluate the tumor antigen binding property, 16E3H3 M1 clone was analyzed for its binding to B7-H4-expressing mammalian cells by FACS. Briefly, B7-H4 positive cell (MX-1, CAMA-1, SK-BR3 and OVCAR-3) or B7-H4 negative cells (PANC-1) were incubated with M40413 antibody. After wash by FACS buffer (1% BSA in PBS), the FITC-anti-human IgG 1 antibody was added to each well and incubated at 4° C. for 45 min. The MFI of FITC was evaluated by FACS Calibur. As shown in FIG. 3, 16E3H3 M1 clonebound to B7-H4 positive cancer cell lines in dose-dependent manner.

### Experimental Example 3. T Cell Checkpoint Ligand Activity of Anti-B7-H4 Monospecific Antibody as Measured by IFN-γ Production

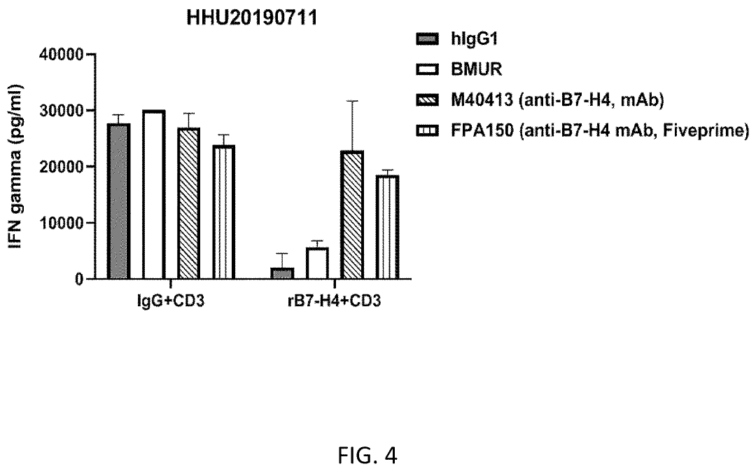

B7-H4 inhibition of T-cell activation was conducted using plate-immobilized recombinant B7-H4 protein (rB7-H4) and PBMC from healthy donor. Normal donor PBMC cells were stimulated with anti-CD3 antibody with IgG, BMUR (Urelumab, anti-4-1BB antibody from BMS), M40413 (16E3H3 M1) or reference B7-H4 antibody (FPA150, Reference antibody from fiveprime) in the presence of control protein (hIgG1) or soluble rB7-H4 (FIG. 4).

rB7-H4 clearly demonstrated T cell checkpoint ligand activity. This can be seen from the graph in FIG. 4 showing that IFN-γsecretion from T cells in rB7-H4-coated plate has been reduced when compared to those in IgG-coated plate. On the other hand, M40413 (graph in Slash pattern) and FPA150 (graph in Vertical pattern) blocked the T cell checkpoint ligand activity of rB7-H4, which can be supported by the increase in IFN-γ secretion level compared with IgG (graph in gray) or BMUR (graph in White) treated condition.

That is, in B7-H4 mediated immune suppressive condition, other immune modulators such as an anti-4-1BB antibody does not function. In contrast, such B7-H4 mediated immune suppression could be overcome by anti-B7-H4 antibody. In together, B7-H4 mediated T-cell suppression was rescued more efficiently by B7-H4 antibody of the present disclosure than antibodies which are not specific to B7-H4 (T-cell activity recovery rate: M40413>FPA150>BMUR).

### Experimental Example 4. T Cell Checkpoint Ligand Activity of Anti-B7-H4 Antibody with Dose Dependent Manner

In order to characterize the T cell checkpoint blockade activity of the B7-H4 antibodies to stimulated human peripheral blood mononuclear cells (PBMCs) response, the concentration of IFN-gamma in supernatant secreted from T cell was measured. B7-H4 inhibition of T-cell activation was conducted using plate-immobilized recombinant B7-H4 protein and PBMC from healthy donor. Various concentrations of Human IgG1, BMUR (anti-4-1BB antibody from BMS) and B7-H4 antibodies—M40413 or FPA150—were incubated with PBMC in the presence of anti-human CD3 to be tested. After culture in a humidified chamber with 5% CO₂ at 37° C. for 72 hours, the concentration of IFN-gamma in supernatant was measured by Human IFN-gamma Quantikine Kit (R&D system, SIF50).

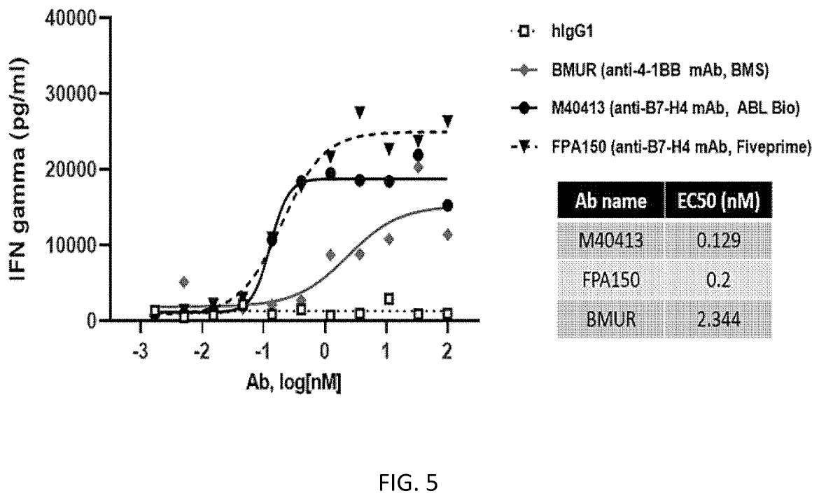

As shown in FIG. 5, M40413 (graph in circle) and FPA150 (graph in inverted triangle) blocked the T cell checkpoint ligand activity of rB7-H4, which can be supported by the increase in IFN-γsecretion level compared with IgG (graph in empty square) or BMUR (graph in rhombus) treated condition. That is, in B7-H4 mediated immune suppressive condition, other immune modulators such as an anti-4-1BB antibody does not function. In contrast, such B7-H4 mediated immune suppression could be overcome by anti-B7-H4 antibody. In together, B7-H4 mediated T-cell suppression was rescued more efficiently by B7-H4 antibody of the present disclosure than antibodies which are not specific to B7-H4.

### Experimental Example 5. Estimation of Relative Expression of Cell Surface B7-H4 Using Dako's Antibody Capacity Kit

Cell surface B7-H4 expression level was quantified on various cancer cell lines using QIFIKIT quantification kit (Dako) according to manufacturer's recommendation. Briefly, cells were stained with unlabeled anti-B7-H4 mouse monoclonal antibody (abcam) or purified mouse IgG1k isotype control (abcam) at saturating concentration. After washing, the stained cells and calibration beads from the kit were simultaneously labeled with the same FITC-conjugated goat anti-mouse IgG secondary antibody from the kit. Labeled cells and calibration beads were analyzed on a flow cytometer. A linear regression was performed using MFI values from the calibration beads. ABC (Antibody-Binding Capacity) was extrapolated from this regression line and sABC (specific ABC) was determined by subtracting ABC of the isotype control antibody from ABC of anti-B7-H4 antibody. As shown in Table 25, the sABC of 11 cancer cell lines was determined. CHOK1, PANC1 and MC38 was regarded as B7-H4 negative cell lines.

TABLE 25

| | | | | Fold of |
|---|---|---|---|---|
| | | | EC50 (nM) | Induction |
| | SABC* | MFI | [NF-kB | [NF-kB |
| Cell line | [B7-H4] | [B7-H4] | assay] | assay] |
| B-CAG-hB7-H4 MC38 | 284,173 | 722 | 0.093 | 76.08 |
| CAMA-1 | 47,564 | 135 | 0.145 | 44.26 |
| MX-1 | 43,245 | 127 | 0.184 | 62.90 |
| SK-BR3 | 29,850 | 79.9 | 0.234 | 30.79 |
| MDA-MB-468 | 13,984 | 41 | 0.403 | 6.74 |
| OVCAR3 | 671 | 7.7 | 0.288 | 5.02 |
| HCC1954 | 411 | 9.6 | 0.647 | 3.01 |
| CHOK1 | 64 | 3.7 | Ambiguous | 1.49 |
| PANC1 | 10 | 6.9 | Ambiguous | 1.11 |
| MC38 | 0 | 3.8 | Ambiguous | 2.32 |

*SABC: Specific Antibody-Binding Capacity

### Experimental Example 6. Antigen Binding Abilities of Anti-B7-H4/Anti-4-1BB Bispecific Antibodies to Human B7-H4 and 4-1BB Proteins

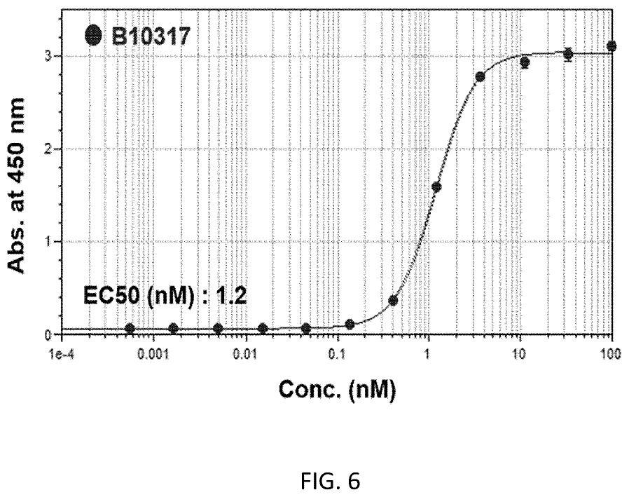

To evaluate the antigen binding activity, the bispecific antibody B10317 (16E3H3 M1×1A10M12) was subjected to DACE (Dual Antigen Captured ELISA). Briefly, microtiter plates were coated with human 4-1BB-Fc protein at 1 μg/ml in PBS, 100 μl/well at 4° C. overnight, then blocked with 200 μl/well of 1% BSA in PBS at 37° C. Three-fold dilutions of bispecific antibodies starting from 100 nM were added to each well and incubated for 1 hours at 37° C. The plates were washed with PBS/Tween and then incubate with 1% BSA in PBS contained human B7-H4 his protein 1 μg/ml for 1 hours at 37° C. The plates were washed with PBST (0.05% Tween 20 in PBS) and then incubated with HRP (Horse Radish Peroxidase) conjugated anti-his antibody for 1 hr at 37° C. After washing, the plates were developed with TMB substrate and analyzed by spectrophotometer at 450 nm˜650 nm. As shown in FIG. 6, B10317 bound to both of human B7-H4 and human 4-1BB at the same time with dose dependent manner. EC₅₀ (nM) value was 1.2 nM.

### Experimental Example 7. Antigen Binding Abilities of Anti-B7-H4/Anti-4-1BB Bispecific Antibodies to Monkey B7-H4 and 4-1BB Proteins

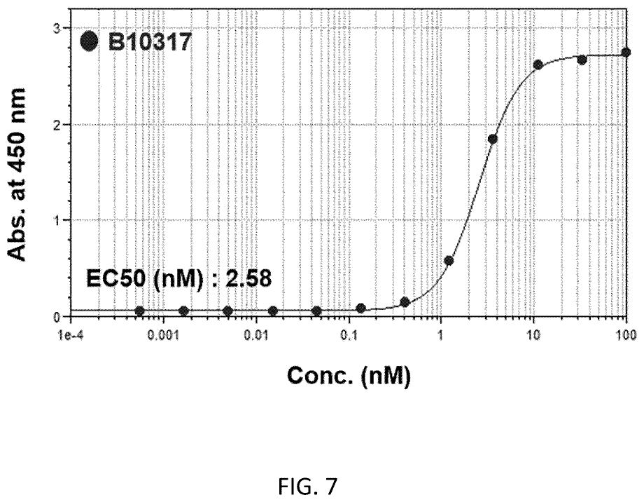

To evaluate the cross reactivity of the bispecific antibody, the bispecific antibody was subjected to DACE. Briefly, microtiter plates were coated with Rhesus 4-1BB-Fc protein (100 ng/well) at 4° C. overnight, then blocked with 200 μl/well PBSB (1% BSA in PBS). Three-fold dilutions of B10317 from 100 nM were added to each well and incubated at 37° C. for 1 hours. The plates were washed with PBST (0.05% Tween20 in PBS) and then incubate for 1 hours at 37° C. with Rhesus B7-H4-his protein (100 ng/well). The plates were washed with PBST (0.05% Tween20 in PBS) and then incubated with HRP (Horse Radish Peroxidase) conjugated Anti-his antibody for 1 hr at 37° C. After washing, the plates were developed with TMB substrate and analyzed by spectrophotometer at 450 nm˜650 nm. As shown in FIG. 7, B10317 bound to Rhesus B7-H4 and 4-1BB simultaneously with dose dependent manner. EC₅₀ (nM) value was 2.58 nM.

### Experimental Example 8. Cell Binding Ability of Anti-B7-H4/Anti-4-1BB Bispecific Antibody

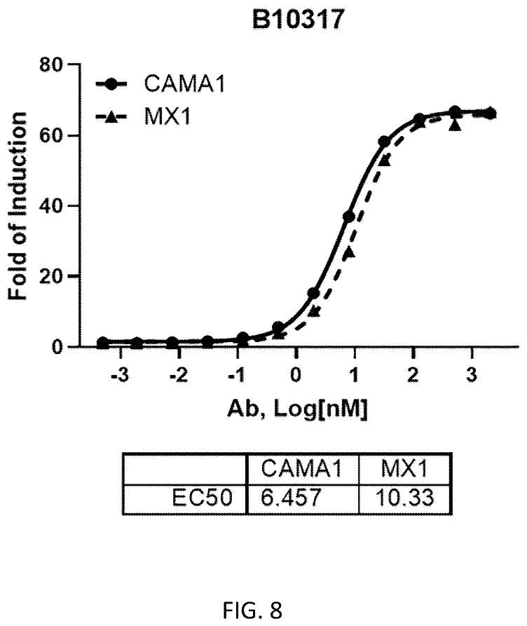

To evaluate the tumor antigen binding property, B10317 bispecific antibody was analyzed for its binding to B7-H4-expressing mammalian cells by FACS. Briefly, B7-H4 positive cells (MX-1 and CAMA-1) were incubated with B10317 antibodies. After wash by FACS buffer (1% BSA in PBS), the FITC-anti-human IgG antibody was added to each well and incubated at 4° C. for 45 min. The MFI of FITC was evaluated by FACS Calibur. As shown in FIG. 8, B10317 bound to B7-H4 positive cancer cell lines in dose-dependent manner.

### Experimental Example 9. Binding Affinity of Anti-B7-H4/Anti-4-1BB Bispecific Antibody to Target Proteins B7-H4 and 4-1BB (SPR)

In the SPR experiment, the anti-B7-H4/anti-4-1BB bispecific antibody obtained in Preparation Example 3 was captured on flow-cells 2, 3 and 4, keeping the flow-cell 1 as reference, on a Protein A Chip on which an anti-B7-H4/anti-4-1BB Bispecific antibody (B10317) has been immobilized by amine coupling. Recombinant B7-H4 (Human/Monkey) or 4-1BB (Human/Monkey) protein was flowed across the chip at concentration range from 100 nM to 6.25 nM for B7-H4 or 250 nM to 15.625 nM for 4-1BB at 30 μl/min for 60 seconds, followed by a dissociation phase of 180 seconds. Regeneration was performed with 10 mM Glycine-HCl (pH 1.5). The obtained results are shown in following Table 26. As shown in Tables 26, B10317 showed high affinity against B7-H4 and 4-1BB, and its affinities for each human and monkey target (B7-H4 or 4-1BB) are comparable.

TABLE 26

| | | Ka | Kd | KD | Rmax |
|---|---|---|---|---|---|
| Target | Species | (1/Ms) | (1/s) | (M) | (RU) |
| B7-H4 | Human | 5.169 × 10⁵ | 2.254 × 10⁻³ | 4.361 × 10⁻⁹ | 13.79 |
| | Monkey | 6.145 × 10⁵ | 2.782 × 10⁻³ | 4.527 × 10⁻⁹ | 13.60 |
| 4-1BB | Human | 2.132 × 10⁵ | 9.706 × 10⁻⁴ | 4.555 × 10⁻⁹ | 58.84 |
| | Monkey | 2.239 × 10⁵ | 8.986 × 10⁻⁴ | 4.015 × 10⁻⁹ | 57.93 |

### Experimental Example 10. Protein Binding Test Against B7-Family Proteins

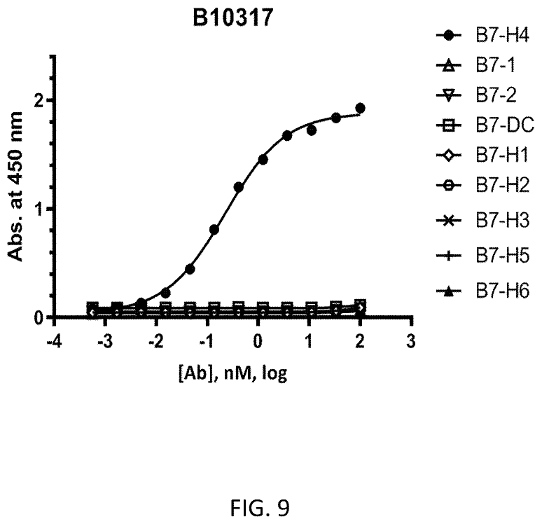

To confirm the specific binding of anti-B7-H4/anti-4-1BB bispecific antibody against B7-H4, B10317 subjected to ELISA test. Microtiter plates were coated with each protein (100 ng/well) at 4° C. overnight, then blocked with 200 μl/well PBSB (1% (w/v) BSA in PBS). Three-fold dilutions of B10317 starting from 100 nM was added to each well and incubated at 37° C. for 1 hours. The plates were washed with PBST (0.05% (v/v) Tween20 in PBS) and then incubate for 1 hours at 37° C. with 100 ul of Anti-Fc HRP (Dilution factor, 1:30000) or Anti-Fab HRP (Dilution factor, 1:20000). The plates were washed with PBST (0.05% (v/v) Tween20 in PBS) and then the plates were developed with TMB substrate and analyzed by spectrophotometer at 450-650 nm. As shown in FIG. 9, it was confirmed that B10317 specifically bound to B7-H4 and did not bind to other B7-family proteins.

### Experimental Example 11. T Cell Checkpoint Ligand Activity of Anti-B7-H4/Anti-4-1BB Bispecific Antibody as Measured by IFN-γ Production

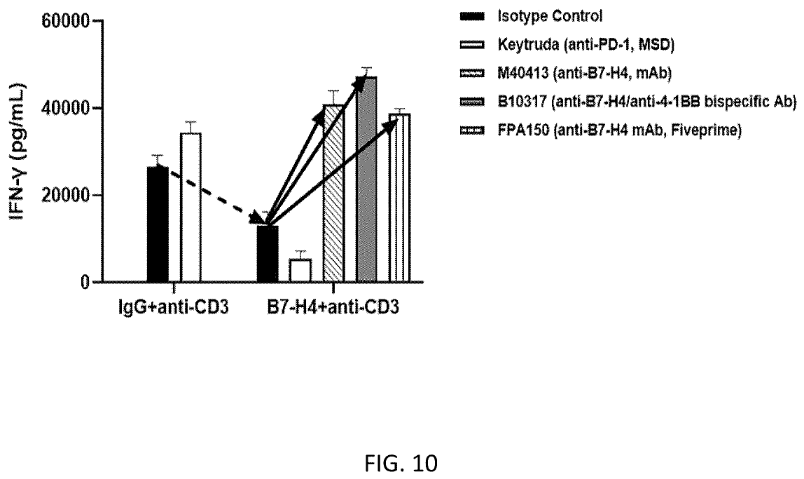

B7-H4 inhibition of T-cell activation was conducted using plate-immobilized recombinant B7-H4 protein (rB7-H4) and PBMC from healthy donor. Normal donor PBMC cells were stimulated with anti-CD3 antibody with IgG, B10317 bispecific antibody, Keytruda (anti-PD-1 antibody from MSD), M40413 monospecific antibody or reference B7-H4 antibody in the presence of control protein (hIgG1) or soluble rB7-H4 (FIG. 10).

rB7-H4 clearly demonstrated T cell checkpoint ligand activity. As shown FIG. 10, IFN-γ secretion from T cells in rB7-H4-coated plate was reduced when compared to those in IgG-coated plate. On the other hand, B10317 (graph in Grey), M40413 (graph in Slash pattern) and FPA150 (graph in Vertical pattern) blocked the T cell checkpoint ligand activity of rB7-H4, which can be supported by the increase in IFN-γ secretion level compared with IgG (graph in Black) or Keytruda (graph in White) treated condition.

That is, in B7-H4 mediated immune suppressive condition, other immune modulators such as an anti-PD-1 antibody does not function. In contrast, such B7-H4 mediated immune suppression could be overcome by anti-B7-H4 antibody according to the present disclosure. In together, B7-H4 mediated T-cell suppression was rescued more efficiently by monospecific or bispecific B7-H4 antibodies of the present disclosure than the control antibodies (T-cell activity recovery rate: B10317>M40413≈FPA150).

### Experimental Example 12. T Cell Checkpoint Ligand Activity of Anti-B7-H4/Anti-4-1BB Bispecific Antibody with Dose Dependent Manner in Various PBMCs Lot Co-Culture System

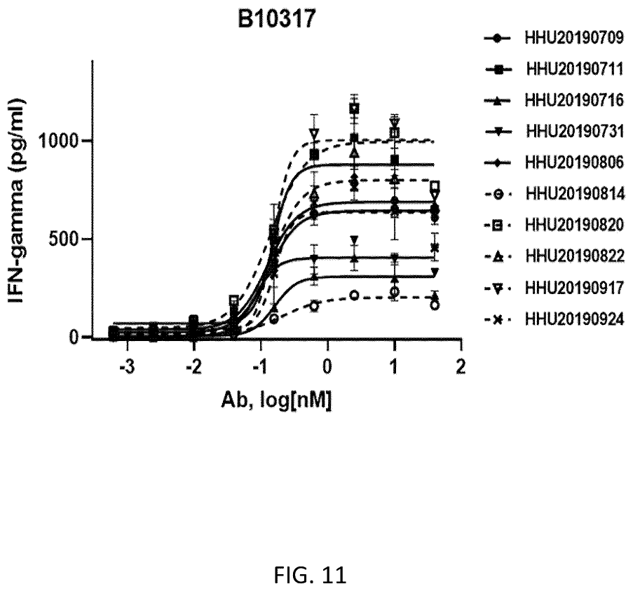

In the same method as Experimental Example 4, for confirming the T cell checkpoint blockade activity of B10317, the concentration of IFN-gamma in supernatant were measured in various PBMCs lot co-culture system. As shown FIG. 11, although the level of IFN-gamma in supernatant was different according to the PBMC lot, the level of IFN-gamma secretion was increased in a dose-dependent manner.

### Experimental Example 13. PBMC Based Target Cell Lysis Test of Anti-B7-H4/Anti-4-1BB Bispecific Antibody

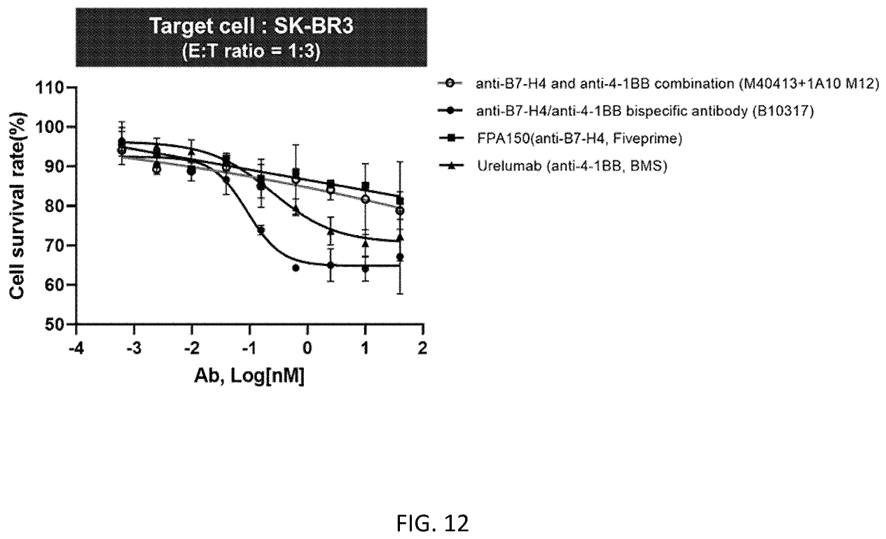

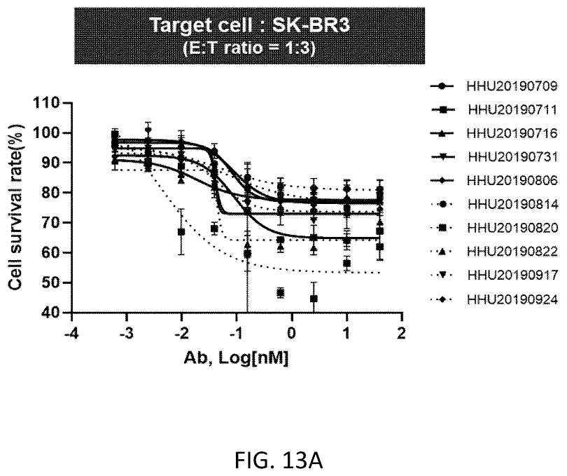

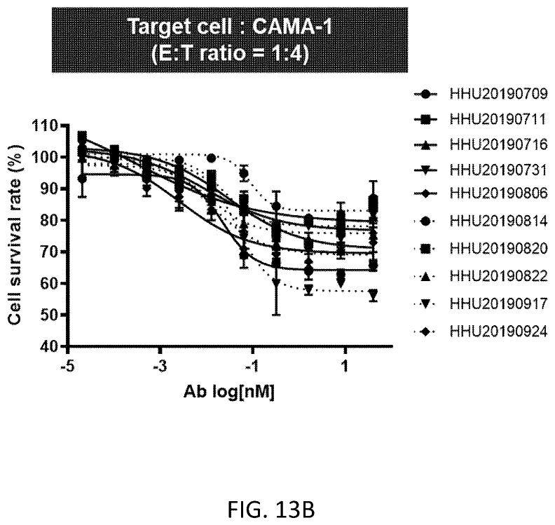

Human PBMCs were co-cultured with SK-BR3 cell line (expressing human B7-H4) in the presence of anti-human CD3 antibody and test antibodies. In brief, PBMCs (Lot No. HHU20190711) were plated with 3.0×10⁴ cells per well and SK-BR3 were co plated with 1.0×10⁴ cells per well (E:T ratio=1:3). Bispecific antibody B10317 (starting from 40 nM diluted for 4-fold), reference antibody FPA150, Urelumab (anti-4-1BB mAb from BMS), or combined treatment group of anti-B7-H4 (M40413) and anti-4-1BB (1A10M12) monospecific antibodies (starting from 40 nM diluted for 4-fold) were each added to the plate well. After 6 days culture, % of target cell lysis was measured by cell counting kit-8 (CCK-8, Dojindo_CK04-20). As shown in FIG. 12, anti-B7-H4/anti-4-1BB antibody (B10317) induced dose-dependent target cell lysis more effectively than Urelumab (Agonistic anti-4-1BB antibody), FPA150 or combined treatment group (combination of anti-B7-H4 and anti-4-1BB monospecific antibodies). Using the same method as above, the target cell lysis ability of B10317 was tested using various PBMC lots, and the results are shown in FIG. 13A (SK-BR3) and FIG. 13b (CAMA-1). As shown in FIGS. 13A and 13B, B10317 induced dose-dependent target cell lysis even when various PBMC lots were used.

### Experimental Example 14. In Vitro 4-1BB Activation Test of the Bispecific Antibody in Various B7-H4 Expressed Condition

Anti-B7-H4/4-1BB bispecific antibody B10317 was analyzed for its in vitro 4-1BB activity by using Promega kit system. In brief, plate various cell lines (MC38-hB7-H4: 2.5×10⁴, CAMA-1: 2.5×10⁴, SK-BR3: 2.5×10⁴, HCC1806-hB7-H4: 2.5×10⁴, OVCAR3: 2.5×10⁴, HCC1954: 2.5×10⁴, CHOK1: 2.5×10⁴, Panc-1: 2.5×10⁴ and MC38: 2.5×10⁴) in a white 96-well assay plate in 100 μL culture medium each.

Cells were cultured overnight in 5% CO₂ humidified 37° C. incubator. After overnight culture, 100 μL of culture medium was removed and 25 μL of assay medium (1% FBS containing RPMI1640) was added to pre-plate target cells. 25 μL of B10317 (starting from 50 nM diluted for 3-fold) or BMUR (starting from 133 nM diluted for 6-fold) was added to the plate. GloResponse™ NFκB-luc2/4-1BB Jurkat cell line was harvested and resuspend with assay Medium. 25 μL of GloResponse™ NFκB-luc2/4-1BB Jurkat cell line was added to each well to make 2.5×10⁴ cells per well to plate. Cells were cultured for 6 hrs in 5% CO₂ humidified 37° C. incubator. During the incubation, Bio-Glo™ reagent was reconstituted according to the manufacturer's instruction. After 6 hrs incubation, 75 μL per well of Bio-Glo™ Reagent was added to the assay plate. After 5 minutes, luminescence was measured using microplate reader. Four-parameter logistic curve analysis was performed with GraphPad software.

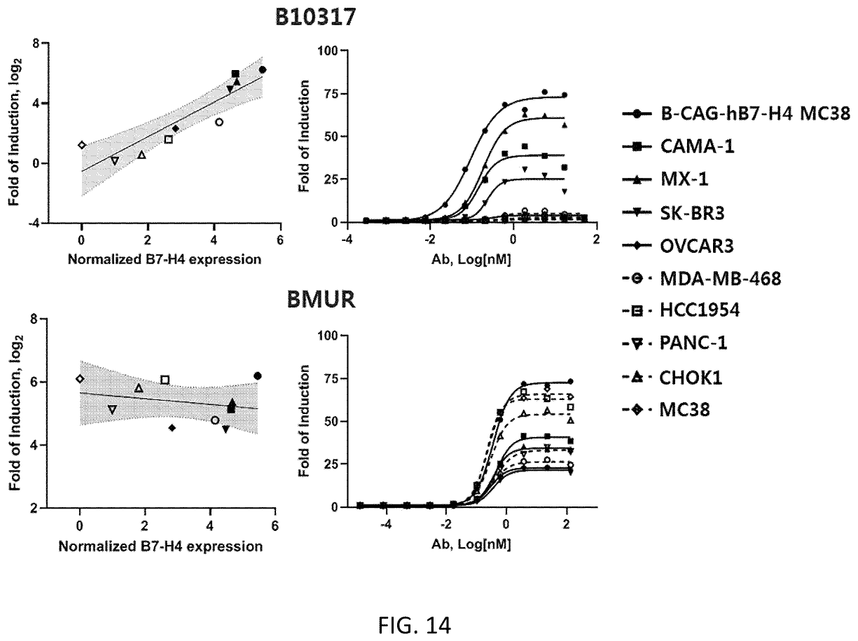

As shown in FIG. 14, B10317 activated 4-1BB signaling only in the presence of B7-H4 with dose dependent manner, and T cell stimulatory activity of B10317 was related with level of B7-H4 expression. B10317 did not activate 4-1BB signaling in the absence of B7-H4 expression. On the other hand, in the BMUR treatment group, T-cell stimulatory activity was increased in both B7-H4 positive and negative cell line. It can be seen from the above results that B10317 especially increases T-cell activity in the B7-H4-expressing tumor environment, differently from BMUR.

### Experimental Example 15. Efficacy Dose Evaluation of the Bispecific Antibody with B-CAG-hB7-H4 MC38 Bearing Humanized 4-1BB Mice

To test the in vivo efficacy of B10317 bispecific antibody, B-h4-1BB TG mice were subcutaneously injected with B-CAG-hB7-H4 MC38 tumor cells (1×10⁶) suspended in 0.1 mL PBS in the right front flank for tumor development. Tumor-bearing animals were randomly enrolled into five study groups when the mean tumor size reached 103 mm³. Each group consisted of 8 mice. The five groups were G1 (hIgG1 isotype control, 10 mg/kg), G2 (BMUR, 7.5 mg/kg, the same molar ratio as 10 mg/kg of B10317), G3 (B10317, 10 mg/kg), G4 (B10317, 2 mg/kg) and G5 (B10317, 0.4 mg/kg). All test articles were intravenously administrated to tumor-bearing mice at a frequency of once every three days for total eight times. The tumor volume and body weight were measured and recorded twice per week. The study was terminated 22 days post last dosing. On day 25 post grouping, the mean tumor volume of G1 (control group) was 1752±269 mm³. The mean tumor volume of G2 group was 48±40 mm³ with 103.3% TGI. In G3, G4 and G5 group, the mean tumor volume was 18±14 mm³ with a TGI of 105.1%, 136±110 mm³ with a TGI of 97.9%, and 470±192 mm³ with a TGI of 77.7%, respectively. The mean body weight and mean tumor volume were shown in Tables 27 and 28. Both BMUR and B10317 did not present any negative affect to the animal body weight or obvious clinical sign (Table 27). The tumor growth inhibition (TGI_{TV}) was calculated and presented in Table 28. Both BMUR and B10317 demonstrated significant anti-tumor activity on tumor volume. In the B10317 treated groups, it showed excellent tumor growth inhibition rate even in the low dose group, demonstrating superior efficacy compared to BMUR.

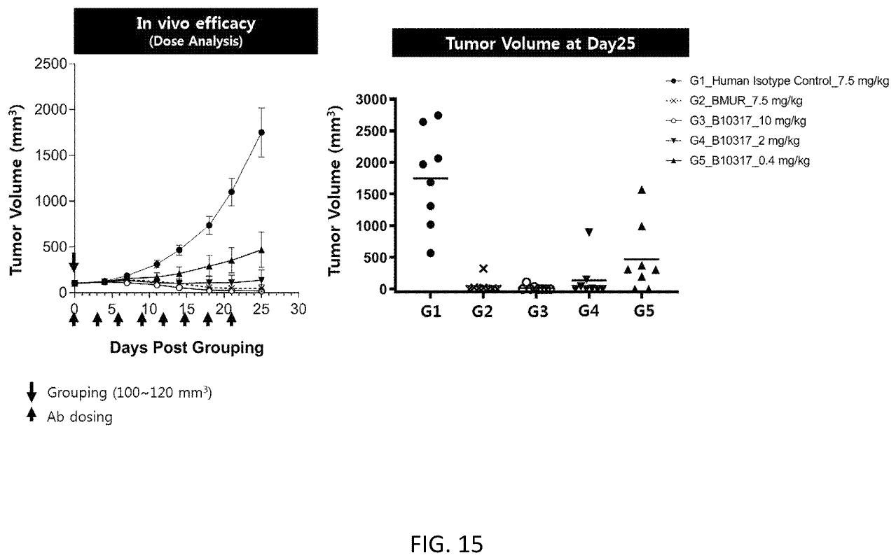

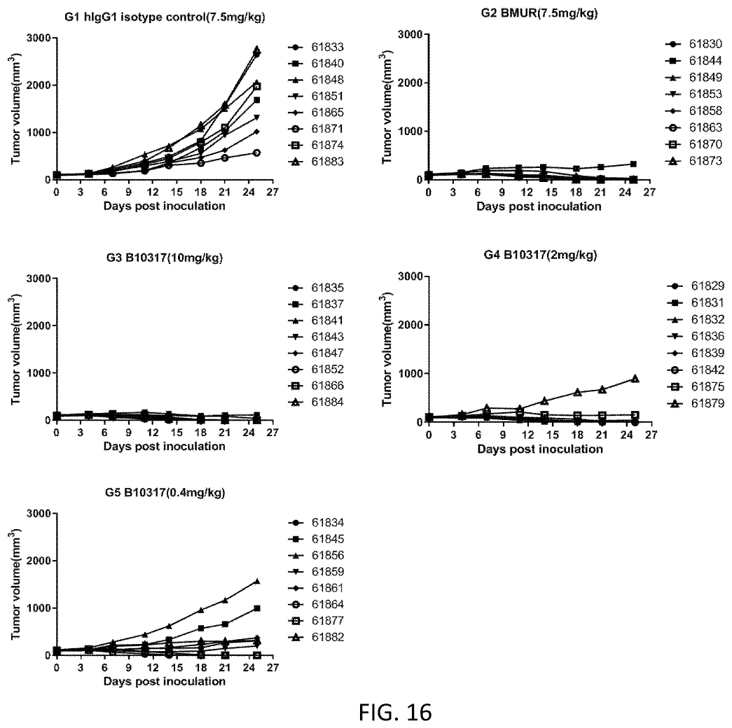

The mean tumor growth profile and volume upon treatment were shown in FIG. 15 and the individual tumor volume were shown in FIG. 16. As shown in FIGS. 15 and 16, B10317 significantly reduced tumor growth in vivo in MC38-hB7-H4 bearing model with dose dependent manner. BMUR treatment group (G2) also showed in vivo anti-tumor activity, but B10317 showed stronger in vivo efficacy (i.e., around 100% of tumor growth inhibition) even in 2 mpk dose (G4).

TABLE 27
Body weight change of B-h4-1BB mice inoculated
with B-CAG-hB7-H4MC38 cells

| | | Body Weight (g) | | | |
|---|---|---|---|---|---|
| | | Before | Day 25 | | Change |
| Group | Test articles | Treatment | Post Grouping | P^{b} | (g) |
| G1 | Isotype | 17.3 ± 0.3 | 19.9 ± 0.6 | — | +2.6 |
| | Control | | | | |
| | (7.5 mg/kg) | | | | |
| G2 | BMUR | 17.4 ± 0.3 | 18.6 ± 0.3 | 0.081 | +1.2 |
| | (7.5 mg/kg) | | | | |
| G3 | B10317 | 17.4 ± 0.2 | 19.1 ± 0.4 | 0.295 | +1.7 |
| | (10 mg/kg) | | | | |
| G4 | B10317 | 17.4 ± 0.3 | 18.7 ± 0.4 | 0.134 | +1.3 |
| | (2 mg/kg) | | | | |
| G5 | B10317 | 17.4 ± 0.4 | 19.4 ± 0.4 | 0.549 | +2.0 |
| | (0.4 mg/kg) | | | | |

^{b}Statistical analysis via t-test on body weight of treatment group versus hIgG1 isotype control group on day 25 post start of treatment.

TABLE 28
Tumor growth inhibition of anti-B7-H4/anti-4-1BB Bispecific
antibodies on B-h4-1BB mice with B-CAG-hB7-H4 MC38 cells

| | | Tumor Volume (mm³) | | | |
|---|---|---|---|---|---|
| | | Before | Day 25 | TGI | |
| Group | Test articles | Treatment | Post Treatment | (%) | p^{b} |
| G1 | Isotype | 103 ± 2 | 1752 ± 269 | — | — |
| | Control | | | | |
| | (7.5 mg/kg) | | | | |
| G2 | BMUR | 103 ± 3 | 48 ± 40 | 103.3 | ***<0.001 |
| | (7.5 mg/kg) | | | | |
| G3 | B10317 | 103 ± 3 | 18 ± 14 | 105.1 | ***<0.001 |
| | (10 mg/kg) | | | | |
| G4 | B10317 | 103 ± 2 | 136 ± 110 | 97.9 | ***<0.001 |
| | (2 mg/kg) | | | | |
| G5 | B10317 | 103 ± 3 | 470 ± 192 | 77.7 | **0.002 |
| | (0.4 mg/kg) | | | | |

a: Mean ± SEM
^{b}Statistical analysis via t-test on body weight of treatment group versus hIgG1 isotype control group on day 25 post start of treatment.
**P < 0.01;
***P < 0.001.

### Experimental Example 16. The Bispecific Antibody Completely Protected Mice from Re-Challenge with MC38-hB7-H4

On day 22 after final treatment, 21 cured mice (seven mice for G2, seven mice for G3, five mice for G4 and two mice for G5) in administrated groups and 5 naive mice were implanted tumor cells for re-challenge experiment. Mice were subcutaneously injected with B-CAG-hB7-H4 MC38 colon tumor cells (1×10⁶) in the left front flank for tumor development.

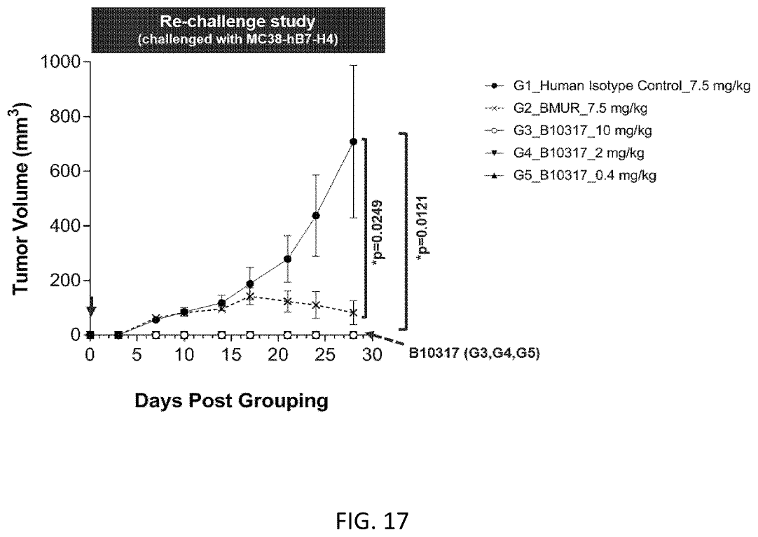

As shown FIG. 17, both BMUR and B10317 demonstrated significant anti-tumor activity under the tested doses and presented no negatively affecting the animal body weight or inducing any obvious clinical sign.

There was no tumor growth with B-CAG-hB7-H4 MC38 re-inoculated in the B10317 treatment group (G3, G4 and G5), it promoted long-term memory protection against B-CAG-hB7-H4 MC38 tumors. Two out seven tumors grown up slowly in BMUR treatment cured group (G2), other tumors appeared in the early phase, and almost disappeared on late phase.

In overall, B10317 showed more potent long-term memory protection than BMUR against B-CAG-hB7-H4 MC38 tumor.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1-19. (canceled)

20. An anti-B7-H4/anti-4-1BB bispecific antibody comprising an anti-B7-H4 antibody or an antigen-binding fragment thereof and an anti-4-1BB antibody or an antigen-binding fragment thereof,
wherein the anti-B7-H4 antibody or the antigen-binding fragment thereof comprises a heavy chain variable region and a light chain variable region comprising:
a heavy chain complementarity-determining region 1 (CDR-H1) comprising an amino acid sequence selected from the group consisting of SEQ ID NOs: 2, 25 and 33;
a CDR-H2 comprising an amino acid sequence selected from the group consisting of SEQ ID NOs: 3, 26 and 34;
a CDR-H3 comprising an amino acid sequence selected from the group consisting of SEQ ID NOs: 4, 27 and 35;
a light chain complementarity-determining region 1 (CDR-L1) comprising an amino acid sequence selected from the group consisting of SEQ ID NOs: 7, 8, 29 and 37;
a CDR-L2 comprising an amino acid sequence selected from the group consisting of SEQ ID NOs: 9 and 30; and
a CDR-L3 comprising an amino acid sequence selected from the group consisting of SEQ ID NOs: 10, 31 and 38; and
wherein the anti-4-1BB antibody or the antigen-binding fragment thereof comprises a heavy chain variable region comprising at least one amino acid sequence selected from the group consisting of SEQ ID NOs: 39 to 43; and a light chain variable region comprising at least one amino acid sequence selected from the group consisting of SEQ ID NOs: 52 and 53.

21. The anti-B7-H4/anti-4-1BB bispecific antibody of claim 20, wherein the anti-B7-H4 antibody or the antigen-binding fragment thereof comprises:
a heavy chain variable region comprising an amino acid sequence selected from the group consisting of SEQ ID NO: 1, 22, 24 and 32; and
a light chain variable region comprising an amino acid selected from the group consisting of SEQ ID NOs: 5, 6, 23, 28 and 36.

22. The anti-B7-H4/anti-4-1BB bispecific antibody of claim 20, wherein the anti-B7-H4 antibody or the antigen-binding fragment thereof comprises:
a heavy chain framework 1 (H-FR1) comprising an amino acid sequence of SEQ ID NO: 12;
a heavy chain framework 2 (H-FR2) comprising an amino acid sequence of SEQ ID NO: 13;
a heavy chain framework 3 (H-FR3) comprising an amino acid sequence of SEQ ID NO: 14;
a heavy chain framework 4 (H-FR4) comprising an amino acid sequence of SEQ ID NO: 15;
a light chain framework 1 (L-FR1) comprising an amino acid sequence of SEQ ID NO: 17;
a light chain framework 2 (L-FR2) comprising an amino acid sequence of SEQ ID NO: 18;
a light chain framework 3 (L-FR3) comprising an amino acid sequence of SEQ ID NO: 19; and
a light chain framework 4 (L-FR4) comprising an amino acid sequence of SEQ ID NO: 20.

23. The anti-B7-H4/anti-4-1BB bispecific antibody of claim 20, wherein each of the anti-B7-H4 antibody or the antigen-binding fragment thereof and the anti-4-1BB antibody or the antigen-binding fragment thereof is independently a chimeric antibody, a humanized antibody, or a human antibody.

24. An isolated polynucleotide encoding the antibody or the antigen-binding fragment according to claim 20.

25. A method of preventing or treating a disease related to B7-H4, 4-1BB, or both thereof in an individual, the method comprising administering the anti-B7-H4/anti-4-1BB bispecific antibody or the antigen-binding fragment of claim 20 to the individual.

26. The method of claim 25, wherein the disease related to B7-H4, 4-1BB, or both thereof is cancer.

27. The method of claim 26, wherein the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer, and thyroid cancer.

28. An anti-B7-H4 antibody or an antigen-binding fragment thereof, having specificity to a B7-H4 protein, wherein the anti-B7-H4 antibody or the antigen-binding fragment thereof comprises a heavy chain variable region and a light chain variable region comprising:
a heavy chain complementarity-determining region 1 (CDR-H1) comprising an amino acid sequence selected from the group consisting of SEQ ID NOs: 2, 25 and 33;
a CDR-H2 comprising an amino acid sequence selected from the group consisting of SEQ ID NOs: 3, 26 and 34;
a CDR-H3 comprising an amino acid sequence selected from the group consisting of SEQ ID NOs: 4, 27 and 35;
a light chain complementarity-determining region 1 (CDR-L1) comprising an amino acid sequence selected from the group consisting of SEQ ID NOs: 7, 8, 29 and 37;
a CDR-L2 comprising an amino acid sequence selected from the group consisting of SEQ ID NOs: 9 and 30; and
a CDR-L3 comprising an amino acid sequence selected from the group consisting of SEQ ID NOs: 10, 31 and 38.

29. The anti-B7-H4 antibody or the antigen-binding fragment of claim 28 comprising:
a heavy chain variable region comprising an amino acid sequence selected from the group consisting of SEQ ID NO: 1, 22, 24 and 32; and
a light chain variable region comprising an amino acid selected from the group consisting of SEQ ID NOs: 5, 6, 23, 28 and 36.

30. The anti-B7-H4 antibody or the antigen-binding fragment of claim 28, which is a chimeric antibody, a humanized antibody, or a human antibody.

31. An isolated polynucleotide encoding the antibody or the antigen-binding fragment according to claim 28.

32. A method of preventing or treating a disease related to B7-H4 in an individual, the method comprising administering the anti-B7-H4 antibody or the antigen-binding fragment of claim 28 to the individual.

33. The method of claim 32, wherein the disease related to B7-H4 is cancer.

34. The method of claim 33, wherein the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer, and thyroid cancer.
